(19) United States
(12) Patent Application Publication
(10) Pub. No.: US 2024/0043830 A1
(43) Pub. Date: Feb. 8, 2024

(54) MULTIPLEX EPIGENOME EDITING

(71) Applicant: The Trustees of Columbia University in the City of New York, New York, NY (US)

(72) Inventor: X. Shawn LIU, New York, NY (US)

(21) Appl. No.: 18/312,865

(22) Filed: May 5, 2023

Related U.S. Application Data
(63) Continuation of application No. PCT/US21/58938, filed on Nov. 11, 2021.
(60) Provisional application No. 63/174,297, filed on Apr. 13, 2021.
(60) Provisional application No. 63/112,331, filed on Nov. 11, 2020.

(51) Int. Cl. C12N 15/10; C12N 9/22; C12N 15/113; C12N 15/86; A61K 48/00; C12N 5/0793; A61K 35/30; A61P 25/00; C12N 15/90

(52) U.S. Cl. CPC C12N 15/102; C12N 9/22; C12N 15/113; C12N 15/86; A61K 48/005; C12N 5/0619; A61K 35/30; A61P 25/00; C12N 15/907

(57) ABSTRACT

The present disclosure provides for systems and methods for modifying the epigenome of cells.

### CROSS REFERENCE TO RELATED APPLICATION

This application is a continuation application of PCT Application No. PCT/US21/58938 filed on Nov. 11, 2021, which claims priority to U.S. Provisional Application No. 63/112,331 filed on Nov. 11, 2020, and U.S. Provisional Application No. 63/174,297 filed on Apr. 13, 2021, each of which is incorporated by reference herein in its entirety.

### SEQUENCE LISTING

The instant application contains a Sequence Listing which has been submitted electronically in XML format and is hereby incorporated by reference in its entirety. Said XML copy, created on Sep. 8, 2023, is named 01001_009113-US2_SL.xml and is 61,353 bytes in size.

### FIELD OF THE INVENTION

The present disclosure relates to systems and methods to modify the epigenome of cells.

### BACKGROUND OF THE DISCLOSURE

Traditionally, epigenetics referred to the study of heritable changes of gene expression in the absence of altering the DNA sequence during cell proliferation and development. This definition is rapidly evolving with the progression in the understanding of molecular mechanisms, including, but not limited to, DNA methylation, histone modifications, noncoding RNA, and 3D chromatin structures, responsible for a variety of epigenetic phenotypes observed in monocellular organisms such as yeast to multicellular organisms like humans (Deichmann, U. (2016) Epigenetics: the origins and evolution of a fashionable topic. Dev. Biol. 416, 249-254). It was proposed that epigenetic mechanisms enable the genome to integrate both developmental and environmental signals (Jaenisch et al. (2003) Epigenetic regulation of gene expression: how the genome integrates intrinsic and environmental signals. Nat. Genet. 33, 245-254).

Genetic studies of epigenetic modifiers such as DNA methyltransferases and histone acetyltransferases have revealed a critical role for epigenetic regulation during development and function. Alteration of epigenetic modifications have been documented in a variety of disorders, including neurological disorders (such as neurodevelopmental, psychiatric, and neurodegenerative diseases), cancer and cardiovascular diseases.

The Clustered Regularly Interspaced Short Palindromic Repeats (CRISPR)-Cas system is a prokaryotic immune system that confers resistance to foreign genetic elements such as plasmids and bacteriophages. The CRISPR/Cas9 system exploits RNA-guided DNA-binding and sequence-specific cleavage of a target DNA. A guide RNA (gRNA) can be complementary to a target DNA sequence upstream of a PAM (protospacer adjacent motif) site. The Cas (CRISPR-associated) 9 protein binds to the gRNA and the target DNA and introduces a double-strand break (DSB) in a defined location upstream of the PAM site. Geurts et al., Science 325, 433 (2009); Mashimo et al., PLoS ONE 5, e8870 (2010); Carbery et al., Genetics 186, 451-459 (2010); Tesson et al., Nat. Biotech. 29, 695-696 (2011). Wiedenheft et al. Nature 482, 331-338 (2012); Jinek et al. Science 337, 816-821 (2012); Mali et al. Science 339, 823-826 (2013); Cong et al. Science 339, 819-823 (2013). The ability of the CRISPR/Cas9 system to be programed to cleave not only viral DNA but also other genes opened a new venue for genome engineering. The CRISPR/Cas system has also been used for gene regulation including transcription repression and activation without altering the target sequence.

Development of epigenome editing tools in manipulating gene expression and/or 3D chromatin structures can help modify an epigenome of cells and treat disorders.

### SUMMARY

The present disclosure provides for a system comprising: (a) a first polynucleotide sequence encoding a fusion protein comprising a deoxyribonuclease (DNase) dead Cpf1 (dCpf1) or Cas9 (dCas9) and an effector domain; and (b) a second polynucleotide sequence encoding one or more guide sequences that hybridize to one or more target sequences.

Also encompassed by the present disclosure is a system comprising: (a) a fusion protein comprising a deoxyribonuclease (DNase) dead Cpf1 (dCpf1) or Cas9 (dCas9) and an effector domain, or a first polynucleotide sequence encoding a fusion protein comprising a deoxyribonuclease (DNase) dead Cpf1 (dCpf1) or Cas9 (dCas9) and an effector domain; and (b) one or more guide sequences that hybridize to one or more target sequences, or a second polynucleotide sequence encoding one or more guide sequences that hybridize to one or more target sequences.

In the fusion protein, dCpf1 (or dCas9) is fused with an effector domain directly or indirectly (e.g., through a linker, and/or NLS).

The dCpf1 may be Cpf1 comprising one or more of the following mutations: D908A, E993A, R1226A and D1263A. The dCpf1 may be Cpf1 comprising the following mutation: D833A.

In one embodiment, the dCpf1 is catalytically dead LbCpf1 (from Lachnospiraceae bacterium). In one embodiment, the dCpf1 is LbCpf1 comprising the following mutation: D833A.

In one embodiment, the dCpf1 is catalytically dead AsCpf1 (from Acidaminococcus sp.). In one embodiment, the dCpf1 may be AsCpf1 comprising one or more of the following mutations: D908A, E993A, R1226A and D1263A. In one embodiment, the dCpf1 may be AsCpf1 comprising the following mutations: D908A, E993A, R1226A and D1263A.

The one or more guide sequences may be one or more CRISPR RNA (crRNA) molecules, one or more single-guide RNA (sgRNA) molecules, one or more guide RNA (gRNA) molecules, or combinations thereof.

The first polynucleotide sequence and the second polynucleotide sequence may be on a single vector, or may be on different vectors.

The second polynucleotide sequence may encode two or more, three or more, four or more, five or more, six or more, seven or more, eight or more, nine or more, or ten or more, guide sequences (e.g., crRNA, sgRNA, or gRNA molecules) that hybridize to two or more, three or more, four or more, five or more, six or more, seven or more, eight or more, nine or more, or ten or more, target sequences.

The dCpf1 may have ribonuclease (RNase) activity.

The effector domain may be Tet2, Dnmt3b, CTCF, Tet1, Dnmt3a, or p300. The effector domain may be a portion of Tet2, Dnmt3b, CTCF, Tet1, Dnmt3a, or p300. The effector domain may be a biologically active portion of Tet2, Dnmt3b, CTCF, Tet1, Dnmt3a, or p300.

The effector domain may have an activity to modify an epigenome.

The effector domain may be an enzyme that modifies a histone subunit.

The effector domain may be a histone acetyltransferase (HAT), histone deacetylase (HDAC), histone methyltransferase (HMT), or histone demethylase. For example, the HAT may be p300.

The effector domain may be an enzyme that modifies the methylation state of DNA.

The effector domain may be a DNA methyltransferase (DNMT) or a Ten-Eleven-Translocation (TET) methylcytosine dioxygenase protein. For example, the DNMT protein is Dnmt3b or Dnmt3a. The TET protein may be Tet2 or Tet1.

The effector domain may be CCCTC-binding factor (CTCF). In one embodiment, CTCF is human CTCF. The CTCF may be wild type CTCF or a DNA binding mutant CTCF. The DNA binding mutant CTCF may comprise one or more of the following mutations: K365A, R368A, R396A, and Q418A. The CTCF mutants include, but are not limited to, CTCF(K365A), CTCF(R368A), CTCF(K365A, R368A), CTCF(R396A) and CTCF(Q418A).

The effector domain may be a transcriptional activation domain, such as VP64 and NF-κB p65, or a transcriptional activation domain derived from VP64 or NF-κB p65.

The effector domain may be a transcriptional silencer or transcriptional repression domain. The transcriptional repression domain may be a Krueppel-associated box (KRAB) domain, ERF repressor domain (ERD), or mSin3A interaction domain (SID). The transcriptional silencer may be heterochromatin protein 1 (HP1), or Methyl CpG binding Protein 2 (MeCP2).

The Cpf1 may be from Lachnospiraceae bacterium, Acidaminococcus sp., Flavobacterium brachiophilum, Parcubacteria bacterium, Peregrinibacteria bacterium, Porphyromonas macacae, Lachnospiraceae bacterium, Porphyromonas crevioricanis, Prevotella disiens, Moraxella bovoculi, Leptospira inadai, Francisella novicida, Candidatus methanoplasma termitum, or Eubacterium eligens.

The present disclosure provides for a composition comprising the present system, a cell comprising the present system, and one, two, or more vectors comprising the present system.

The one or more vectors may comprise a recombinant lentiviral vector.

The present disclosure provides for a method for modifying an epigenome of a cell. The method may comprise contacting the cell with the present system.

Also encompassed by the present method for modifying an epigenome of a cell. The method may comprise contacting the cell with a system comprising: (a) a first polynucleotide sequence encoding a fusion protein comprising a deoxyribonuclease (DNase) dead Cpf1 (dCpf1) and an effector domain, where the dCpf1 is Cpf1 comprising (i) one or more of the following mutations: D908A, E993A, R1226A and D1263A, or (ii) the following mutation: D833A; and (b) a second polynucleotide sequence encoding one or more guide sequences that hybridize to one or more target sequences.

The present disclosure provides for a method for modifying an epigenome of a cell. The method may comprise contacting the cell with a system comprising: (a) a fusion protein comprising a deoxyribonuclease (DNase) dead Cpf1 (dCpf1) and an effector domain, or a first polynucleotide sequence encoding a fusion protein comprising a deoxyribonuclease (DNase) dead Cpf1 (dCpf1) and an effector domain; and (b) one or more guide sequences that hybridize to one or more target sequences, or a second polynucleotide sequence encoding one or more guide sequences that hybridize to one or more target sequences.

In certain embodiments, the cell is an induced pluripotent stem cell (iPSC) or a human embryonic stem cell (hESC). For example, the iPSC may be derived from a fibroblast of a subject.

The present method may further comprise culturing the iPSC or hESC to differentiate into a differentiated cell (e.g., a neuron). The present method may further comprise administering the differentiated cell (e.g., neuron) to a subject.

The present disclosure provides for a method for treating a disease in a patient. The method may comprise administering to the patient a system comprising: (a) a first polynucleotide sequence encoding a fusion protein comprising a deoxyribonuclease (DNase) dead Cpf1 (dCpf1) and an effector domain, where the dCpf1 is Cpf1 comprising (i) one or more of the following mutations: D908A, E993A, R1226A and D1263A, or (ii) the following mutation: D833A; and (b) a second polynucleotide sequence encoding one or more guide sequences that hybridize to one or more target sequences.

The present disclosure provides for a method for treating a disease in a patient. The method may comprise administering to the patient a system comprising: (a) a fusion protein comprising a deoxyribonuclease (DNase) dead Cpf1 (dCpf1) and an effector domain, or a first polynucleotide sequence encoding a fusion protein comprising a deoxyribonuclease (DNase) dead Cpf1 (dCpf1) and an effector domain; and (b) one or more guide sequences that hybridize to one or more target sequences, or a second polynucleotide sequence encoding one or more guide sequences that hybridize to one or more target sequences.

The one or more target sequences may be in, or associated with, one or more genes selected from the group consisting of: MECP2, PHEX, COL4A5, COL4A3, COL4A1, IKBKG, PORCN, DMD/DYS, RPS6KA3, LAMP2, NSDHL, PDHA1, HDAC8, SMC1A, CDKL5, OFD1, WDR45, KDM6A, CASK, FINA, ALAS2, HNRNPH2, MSL3 and IQSEC2.

The one or more target sequences may be in, or associated with, one or more genes selected from the genes in Table 1 or Table 2.

In certain embodiments, the disease is a X-linked disease. The X-linked disease may be selected from the diseases in Table 1.

In one embodiment, the disease is Rett syndrome (RTT).

In certain embodiments, the disease is an imprinting-related disease. The imprinting-related disease may be selected from the diseases in Table 2.

The disease may be a neurological disorders (such as a neurodevelopmental disorder, a psychiatric disorder, and a neurodegenerative disorder), cancer, or a cardiovascular diseases.

The present disclosure provides for a system comprising the present polynucleotide(s) and/or components (e.g., protein(s)).

The present disclosure provides for a composition comprising the present system, or a composition comprising the present polynucleotide(s) and/or components (e.g., protein(s)).

The present disclosure provides for a cell comprising the present system, or a cell comprising the present polynucleotide(s) and/or components (e.g., protein(s)).

The present disclosure provides for one or more vectors comprising the present polynucleotide(s) or the present system. In one embodiment, one or more vectors may be a recombinant lentiviral vector.

Also encompassed by the present disclosure is a method for inactivating an endonuclease system in a cell or in a subject. The method may comprise contacting a cell with the present polynucleotide, vector system, or composition. The method may comprise administering to the subject the present polynucleotide, vector, system, or composition.

The present disclosure provides for a method for modifying an epigenome in a cell or in a subject. The method may comprise contacting a cell with the present polynucleotide(s), vector(s), system, or composition. The method may comprise administering to the subject the present polynucleotide(s), vector(s), system, or composition.

The present disclosure provides for a method of treating a condition in a subject. The method may comprise administering to the subject the present polynucleotide(s), vector(s), system, or composition.

### BRIEF DESCRIPTION OF THE DRAWINGS

FIG. 1 is a schematic representation of an “all-in-one” vector (e.g., a plasmid) encoding a crRNA array, Cpf1, and a selection marker.

FIGS. 2A-2C show mutational analysis of Cpf1 with different direct repeats (DR). FIG. 2A shows the structure of Array 1 (Zetsche et al., Cpf1 is a Single RNA-Guided Endonuclease of a Class 2 CRISPR-Cas System, Cell, 2015, 163, 3:759-771; Yamano et al., Crystal Structure of Cpf1 in Complex with Guide RNA and Target DNA, Cell, 2016, 165:949-962) and Array 2 (Zetsche et al., Multiplex gene editing by CRISPR-Cpf1 through autonomous processing of a single crRNA array, Nature Biotechnol. 2017, 35(1): 31-34). FIG. 2B: The ability of Cpf1 with different arrays to induce indels at the DNMT1, VEGFA, GRIN2B targets were examined by the Surveyor assay. Array 1: 19 nucleotide (nt) DR+23 nt guide RNA (gRNA); Array 2: 37 nt DR+23 nt gRNA. Cpf1-TetCD: Cpf1 fused with Tet catalytic domain. FIG. 2C is a Western blot showing the expression levels of Cpf1 and Cpf1-TetCD.

FIG. 3 shows mutational analysis of key residues in the RuvC and Nuc domains of Cpf1. The effects of mutations on the ability of Cpf1 to induce indels at the DNMT1 target were examined by the Surveyor assay.

FIG. 4 shows affinity analysis of key residues in the RuvC and Nuc domains of AsCpf1. Effects of point mutations on the ability of AsCpf1 (DNase activity catalytically dead Cpf1) to bind to the DNMT1, VEGFA and GRIN2B target DNA sequences were examined using chromatin immunoprecipitation (ChIP)-qPCR (n=3, error bars show mean±SEM). Values were normalized against the mock sample.

FIGS. 5A-5B show optimization of the dCpf1-p300 (a catalytic inactive mutant Cpf1 (dCpf1) fused with p300) system to mediate target histone acetylation for gene activation. FIG. 5A shows the relative MyoD mRNA levels normalized against the mock sample. FIG. 5B is a Western blot showing the expression levels of the fusion proteins detected by the anti-HA tag antibodies. dCas9 is Cas9 with the following point mutations: D10A and H840A; dAsCpf1 is AsCpf1 with the following point mutations: D908A, E993A, R1226A and D1263A; dLbCpf1 is LbCpf1 with the following point mutation: D833A. The term “array” refers to crRNA 1-4.

FIG. 6 shows the results to study the effective range of editing H3K27 acetylation at the MyoD locus by the dCpf1-p300 system. dCas9 is Cas9 with the following point mutations: D10A and H840A; dAsCpf1 is AsCpf1 with the following point mutations: D908A, E993A, R1226A and D1263A; dLbCpf1 is LbCpf1 with the following point mutation: D833A.

FIGS. 7A-7B show the results to study the effective range of editing H3K27 acetylation at the MeCP2 locus by the dCpf1-p300 system. FIG. 7A: anti-H3K27Ac antibody was used for ChIP-qPCR. dC: dCdf1. FIG. 7B: anti-HA antibody was used for ChIP-qPCR. dLbCpf1 or dCpf1 is LbCpf1 with the following point mutation: D833A.

FIG. 8 shows that dCpf1-Dnmt3a (dCpf1 fused with Dnmt3a) provides higher DNA methylation editing efficiency than dCas9-Dnmt3a (a catalytic inactive mutant Cas9 (dCas9) fused with Dnmt3a). An all-in-one vector was used which encoded dCpf1-Dnmt3a and crRNA. dCas9 is Cas9 with the following point mutations: D10A and H840A; dCpf1 is LbCpf1 with the following point mutation: D833A.

FIGS. 9A-9C show dCpf1-CTCF can bind to multiple sites. FIG. 9A is a schematic representation of the structure of lentiviral dCpf1-CTCF. FIG. 9B shows the experimental steps. FIG. 9C shows the ChIP-qPCR results using antibodies against Cpf1-HA or CTCF to examine the binding of dCpf1-p300 and dCpf1-CTCF to the targeted MeCP2 locus. dCpf1 is LbCpf1 with the following point mutation: D833A.

FIGS. 10A-10B show that DNA-binding mutants of CTCF (CTCF K365A&R368A; CTCF R396A; CTCF Q418A) reduced the off-target effect of dCpf1-CTCF. FIG. 10A: ChIP-qPCR was performed using anti-HA antibodies to examine the binding of dCpf1-CTCF to the targeted MeCP2 locus. FIG. 10B is a Western blot showing the expression levels of the proteins detected by the anti-HA or anti-CTCF antibodies. dCpf1 is LbCpf1 with the following point mutation: D833A.

FIGS. 11A-11B show dCpf1-CTCF mediated DNA looping of the MeCP2 locus. FIG. 11A shows the ChIP-qPCR results where crRNA-1 was used. FIG. 11B shows the ChIP-qPCR results where crRNA-2 was used.

FIG. 12 is a schematic representation of MECP2 dual color reporter hES cell lines.

FIGS. 13A-13B show demethylation of the Xi-specific DMR at the MECP2 promoter by dCas9-Tet1 (dCas9 fused with Tea). FIG. 13A is a schematic representation of the MECP2 promoter (Lister et al., Global Epigenomic Reconfiguration During Mammalian Brain Development, Science, 2013, 341(6146):1237905) targeted by sgRNAs including sgRNA-1 to sgRNA-10, as well as the regions (Regions a-c) for pyrosequencing (pyro-seq). FIG. 13B shows the pyrosequencing (pyro-seq) results for Regions a-c. dC-T: dCas9-Tet1. dCas9 is Cas9 with the following point mutations: D10A and H840A.

FIG. 14 shows the immunofluorescence images suggesting that methylation editing resulted in reactivation of MECP2 on the inactive X chromosome (Xi) in human embryonic stem cells (hESCs). Cells were infected with lentiviruses expressing dCas9-Tet1-P2A-BFP (dC-T) and lentiviruses expressing sgRNA-mCherry (10 sgRNAs). Fluorescence-activated cell sorting (FACS) was used to isolate cells that were BFP+mCherry+. Infected cells were subject to immunofluorescence staining. dC-T: dCas9-Tet1. dCas9 is Cas9 with the following point mutations: D10A and H840A.

FIG. 15 shows that MECP2 reactivation was maintained in neural precursor cells (NPCs) and neurons. dC-T: dCas9-Tet1. dCas9 is Cas9 with the following point mutations: D10A and H840A. sgRNAs: 10 sgRNAs as discussed above.

FIG. 16 shows that dCas9-Tet1 with a single sgRNA was sufficient to reactivate MECP2 on Xi. MECP2 mutant #860 RTT-like human embryonic stem cells (hESC) were infected with lentiviruses expressing dCas9-Tet1-P2A-BFP (dCas9-Tet1) and lentiviruses expressing sgRNA-mCherry (10 sgRNAs). Fluorescence-activated cell sorting (FACS) was used to isolate cells that were BFP+mCherry+, which were cultured to form ESC colonies. The ESCs were then allowed to differentiate into neurons. The lower panel is Western blot showing the levels of MECP2. dCas9 is Cas9 with the following point mutations: D10A and H840A.

FIGS. 17A-17B show rescue of neuronal soma size in methylation edited neurons. Neurons derived from wild type #38 hESC, mutant #860 RTT-like hESC, and methylation edited #860 were used to examine the soma size by immunofluorescence staining against MECP2 and Map2 (FIG. 17A). The soma sizes were quantified by Image J (FIG. 17B). sgRNAs: 10 sgRNAs as discussed above. dC-T: dCas9-Tet1. dCas9 is Cas9 with the following point mutations: D10A and H840A.

FIGS. 18A-18B show rescue of neuronal activity in methylation edited neurons. Neurons derived from wild type #38 hESC, mutant #860 RTT-like hESC, and methylation edited #860 were used to examine the electrophysical properties post-differentiation by multi-electrode assay (FIG. 18A). FIG. 18B shows the mean firing rates 67 days post-differentiation. sgRNAs: 10 sgRNAs as discussed above. dC-T: dCas9-Tet1. dCas9 is Cas9 with the following point mutations: D10A and H840A.

FIG. 19 shows that MECP2 reactivation was not stable in neurons. Neurons derived from wild type #38 hESC, mutant #860 RTT-like hESC, and methylation edited #860 were infected with lentiviral dCas9-Tet1 and 10 sgRNAs, and the expression of GFP was examined by qPCR. sgRNAs: 10 sgRNAs as discussed above.

FIG. 20 is a schematic representation of the strategy of using dCpf1-CTCF to build an artificial escapee at the MECP2 locus on Xi for reactivation in neurons.

FIGS. 21A-21C show that the combination of methylation editing and DNA looping in RTT neurons rescued the neuronal activity. FIG. 21A shows the targeted CTCF anchor sites in the MECP2 locus. FIG. 21B is a schematic representation of the experimental design. FIG. 21C shows the electrophysical properties of the neurons examined by multi-electrode assay. 10 sgRNAs as discussed above were used. dCas9 is Cas9 with the following point mutations: D10A and H840A; dCpf1 is LbCpf1 with the following point mutation: D833A. dCpf1-CTCF is dCpf1 fused with CTCF.

### DETAILED DESCRIPTION

The present systems can precisely edit the epigenome, including, but not limited to, DNA methylation, histone acetylation, and DNA looping, at one or multiple genomic loci in mammalian cells, both in vitro and in vivo (e.g., in a patient, in animal models such as mice, etc.). The system may comprise a catalytically dead Cpf1 (dCpf1), an orthologue of the CRISPR/Cas9, fused with one or more effector protein/domain, including, but not limited to, Dnmt3a/b, Tet1/2, p300, and CTCF, that can modify the status of DNA methylation, histone acetylation, DNA looping, etc.

Cpf1 may be used in the present methods and systems (Zetsche et al., Cpf1 Is a Single RNA-Guided Endonuclease of a Class 2 CRISPR-Cas System, Cell, 163(3):759-771).

The present disclosure provides for a system comprising: (a) a first polynucleotide sequence encoding a fusion protein comprising a deoxyribonuclease (DNase) dead Cpf1 (dCpf1) and an effector domain, where the dCpf1 is Cpf1 comprising (i) one or more of the following mutations: D908A, E993A, R1226A and D1263A, or (ii) the following mutation: D833A; and (b) a second polynucleotide sequence encoding one or more guide sequences that hybridize to one or more target sequences.

In certain embodiments, the DNase catalytically dead Cpf1 (dCpf1) has RNAse activity.

The target sequence may be located in, or near, a differentially methylated region (DMR), an enhancer, a promoter, and/or a CTCF binding site, of a gene. The target sequence may comprise a DMR, an enhancer, a promoter, and/or a CTCF binding site, of a gene. The one or more target sequences (e.g., genomic sequences) may be located within 50 kB of the transcription start site (TSS) of a gene.

The target sequence may be located in, or near, a differentially methylated region (DMR), an enhancer, a promoter, and/or a CTCF binding site, of a disease associated gene. The target sequence may comprise a DMR, an enhancer, a promoter, and/or a CTCF binding site, of a disease associated gene.

The target sequence may be a genomic sequence. In certain embodiments, 2, 3, 4, 5, 6, 7, 8, 9, 10, 11, 12, 13, 14, 15, 16, 17, 18, 19, or 20 target sequences (e.g., genomic sequences) are modified in the cell.

The present disclosure provides for a system comprising: (a) a fusion protein comprising a deoxyribonuclease (DNase) dead Cpf1 (dCpf1) and an effector domain, where the dCpf1 is Cpf1 comprising (i) one or more of the following mutations: D908A, E993A, R1226A and D1263A, or (ii) the following mutation: D833A, or a first polynucleotide sequence encoding the fusion protein; and (b) one or more guide sequences that hybridize to one or more target sequences, or a second polynucleotide sequence encoding the one or more guide sequences.

In certain embodiments, catalytically inactive Cpf1 (dCpf1) or Cas9 (dCas9) is fused with Tet2, Dnmt3b, CTCF, Tet1, Dnmt3a, or p300. In certain embodiments, targeting of the fusion protein to methylated or unmethylated a promoter, or an enhancer, may activate or silence the expression of a gene. Targeted de novo methylation of a CTCF loop anchor site by the fusion protein may block CTCF binding and interfere with DNA looping, which may alter gene expression in the neighboring loop.

The guide sequence may be a CRISPR RNA (crRNA) molecule, a single-guide RNA (sgRNA) molecule, a guide RNA (gRNA), or combinations thereof.

The first polynucleotide sequence and the second polynucleotide sequence may be on a single vector, or on different vectors.

The second polynucleotide sequence may encode two or more guide sequences that hybridize to two or more target sequences.

In certain embodiments, the system contains an all-in-one vector expressing a chimeric protein (or fusion protein), and one crRNA or an array of crRNAs to target the chimeric protein to one or multiple genomic loci to mediate epigenome editing. Our experimental results show a robust change of epigenetic statuses at the targeted loci. The present method and systems allow exploring the biological functions of multiple epigenetic events and manipulating the disease-associated epigenetic events for the novel therapeutic strategy.

The present disclosure provides for a polynucleotide comprising: (a) a first sequence encoding a fusion protein comprising a catalytically dead or deoxyribonuclease (DNase) dead nuclease and an effector domain; and (b) a second sequence encoding two or more guide sequences that hybridize to two or more genomic sequences.

The nuclease may be a catalytically dead Cpf1 (dCpf1). The nuclease may be a catalytically dead Cas9 (e.g., spCas9). The catalytically dead Cas9 (dCas9) may contain one or more of the following mutations: D10A and H840A. The dCpf1 may comprise one or more of the following mutations: D908A, E993A, R1226A and D1263A. The dCpf1 may be Cpf1 comprising the following mutation: D833A.

The present disclosure provides for a polynucleotide comprising: (a) a first sequence encoding a fusion protein comprising a deoxyribonuclease (DNase) dead Cpf1 (dCpf1) and an effector domain, where the dCpf1 is Cpf1 comprising (i) one or more of the following mutations: D908A, E993A, R1226A and D1263A, or (ii) the following mutation: D833A; and (b) a second sequence encoding two or more guide sequences that hybridize to two or more genomic sequences.

The Cpf1 may be from Flavobacterium brachiophilum, Parcubacteria bacterium, Peregrinibacteria bacterium, Acidaminococcus sp., Porphyromonas macacae, Lachnospiraceae bacterium, Porphyromonas crevioricanis, Prevotella disiens, Moraxella bovoculi, Leptospira inadai, Lachnospiraceae bacterium (MA2020), Francisella novicida, Candidatus methanoplasma termitum, or Eubacterium eligens.

In one embodiment, the dCpf1 is catalytically dead LbCpf1 (from Lachnospiraceae bacterium). In another embodiment, the dCpf1 is catalytically dead AsCpf1 (from Acidaminococcus sp.). In yet another embodiment, the dCpf1 is catalytically dead FbCpf1 (from Flavobacterium brachiophilum).

AsCpf1 may have the UniProt number UniProtKB-U2UMQ6 (CS12A_ACISB), and comprise the corresponding amino acid sequence. LbCpf1 may have the UniProt number UniProtKB-A0A182DWE3 (A0A182DWE3_9FIRM), and comprise the corresponding amino acid sequence.

There may be a number of different isoforms for each of these proteins/polypeptides discussed in this disclosure, provided herein are the general accession numbers, NCBI Reference Sequence (RefSeq) accession numbers, GenBank accession numbers, and/or UniProt numbers to provide relevant sequences. The proteins/polypeptides may also comprise other sequences. In all cases where an accession number (e.g., a UniProt number) are used, the accession number refers to one embodiment of the protein or gene which may be used with the systems/methods of the present disclosure.

AsCpf1 may comprise/have the below amino acid sequence (SEQ ID NO: 43;
Acidaminococcus sp.):
MTQFEGFTNL YQVSKTLRFE LIPQGKTLKH IQEQGFIEED KARNDHYKEL
KPIIDRIYKT YADQCLQLVQ LDWENLSAAI DSYRKEKTEE TRNALIEEQA
TYRNAIHDYF IGRTDNLTDA INKRHAEIYK GLFKAELFNG KVLKQLGTVT
TTEHENALLR SFDKFTTYFS GFYENRKNVF SAEDISTAIP HRIVQDNFPK
FKENCHIFTR LITAVPSLRE HFENVKKAIG IFVSTSIEEV FSFPFYNQLL
TQTQIDLYNQ LLGGISREAG TEKIKGLNEV LNLAIQKNDE TAHIIASLPH
RFIPLFKQIL SDRNTLSFIL EEFKSDEEVI QSFCKYKTLL RNENVLETAE
ALFNELNSID LTHIFISHKK LETISSALCD HWDTLRNALY ERRISELTGK
ITKSAKEKVQ RSLKHEDINL QEIISAAGKE LSEAFKQKTS EILSHAHAAL
DQPLPTTLKK QEEKEILKSQ LDSLLGLYHL LDWFAVDESN EVDPEFSARL
TGIKLEMEPS LSFYNKARNY ATKKPYSVEK FKLNFQMPTL ASGWDVNKEK
NNGAILFVKN GLYYLGIMPK QKGRYKALSF EPTEKTSEGF DKMYYDYFPD
AAKMIPKCST QLKAVTAHFQ THTTPILLSN NFIEPLEITK EIYDLNNPEK
EPKKFQTAYA KKTGDQKGYR EALCKWIDFT RDFLSKYTKT TSIDLSSLRP
SSQYKDLGEY YAELNPLLYH ISFQRIAEKE IMDAVETGKL YLFQIYNKDF
AKGHHGKPNL HTLYWTGLFS PENLAKTSIK LNGQAELFYR PKSRMKRMAH
RLGEKMLNKK LKDQKTPIPD TLYQELYDYV NHRLSHDLSD EARALLPNVI
TKEVSHEIIK DRRFTSDKFF FHVPITLNYQ AANSPSKENQ RVNAYLKEHP
ETPIIGIDRG ERNLIYITVI DSTGKILEQR SLNTIQQFDY QKKLDNREKE
RVAARQAWSV VGTIKDLKQG YLSQVIHEIV DLMIHYQAVV VLENLNFGFK
SKRTGIAEKA VYQQFEKMLI DKLNCLVLKD YPAEKVGGVL NPYQLTDQFT
SFAKMGTQSG FLFYVPAPYT SKIDPLTGFV DPFVWKTIKN HESRKHFLEG
FDFLHYDVKT GDFILHFKMN RNLSFQRGLP GFMPAWDIVF EKNETQFDAK
GTPFIAGKRI VPVIENHRFT GRYRDLYPAN ELIALLEEKG IVFRDGSNIL
PKLLENDDSH AIDTMVALIR SVLQMRNSNA ATGEDYINSP VRDLNGVCFD
SRFQNPEWPM DADANGAYHI ALKGQLLLNH LKESKDLKLQ NGISNQDWLA
YIQELRN

In certain embodiments, AsCpf1 may comprise/have an amino acid sequence at least or about 70%, at least or about 75%, at least or about 80%, at least or about 85%, at least or about 90%, at least or about 95%, at least or about 99%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 43.

In certain embodiments, AsCpf1 may comprise/have an amino acid sequence at least or about 70%, at least or about 75%, at least or about 80%, at least or about 85%, at least or about 90%, at least or about 95%, at least or about 99%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 43, where AsCpf1 contains D908, E993, R1226 and D1263.

LbCpf1 may comprise the below amino acid sequence (SEQ ID NO: 44;
Lachnospiraceae bacterium):
AASKLEKFTN CYSLSKTLRF KAIPVGKTQE NIDNKRLLVE DEKRAEDYKG
VKKLLDRYYL SFINDVLHSI KLKNLNNYIS LFRKKTRTEK ENKELENLEI
NLRKEIAKAF KGAAGYKSLF KKDIIETILP EAADDKDEIA LVNSFNGFTT
AFTGFFDNRE NMFSEEAKST SIAFRCINEN LTRYISNMDI FEKVDAIFDK
HEVQEIKEKI LNSDYDVEDF FEGEFFNFVL TQEGIDVYNA IIGGFVTESG
EKIKGLNEYI NLYNAKTKQA LPKFKPLYKQ VLSDRESLSF YGEGYTSDEE
VLEVFRNTLN KNSEIFSSIK KLEKLFKNFD EYSSAGIFVK NGPAISTISK
DIFGEWNLIR DKWNAEYDDI HLKKKAVVTE KYEDDRRKSF KKIGSFSLEQ
LQEYADADLS VVEKLKEIII QKVDEIYKVY GSSEKLFDAD FVLEKSLKKN
DAVVAIMKDL LDSVKSFENY IKAFFGEGKE TNRDESFYGD FVLAYDILLK
VDHIYDAIRN YVTQKPYSKD KFKLYFQNPQ FMGGWDKDKE TDYRATILRY
GSKYYLAIMD KKYAKCLQKI DKDDVNGNYE KINYKLLPGP NKMLPKVFFS
KKWMAYYNPS EDIQKIYKNG TFKKGDMENL NDCHKLIDFF KDSISRYPKW
SNAYDFNFSE TEKYKDIAGF YREVEEQGYK VSFESASKKE VDKLVEEGKL
YMFQIYNKDF SDKSHGTPNL HTMYFKLLFD ENNHGQIRLS GGAELFMRRA
SLKKEELVVH PANSPIANKN PDNPKKTTTL SYDVYKDKRF SEDQYELHIP
IAINKCPKNI FKINTEVRVL LKHDDNPYVI GIDRGERNLL YIVVVDGKGN
IVEQYSLNEI INNENGIRIK TDYHSLLDKK EKERFEARQN WTSIENIKEL
KAGYISQVVH KICELVEKYD AVIALEDLNS GFKNSRVKVE KQVYQKFEKM
LIDKLNYMVD KKSNPCATGG ALKGYQITNK FESFKSMSTQ NGFIFYIPAW
LTSKIDPSTG FVNLLKTKYT SIADSKKFIS SFDRIMYVPE EDLFEFALDY
KNFSRTDADY IKKWKLYSYG NRIRIFAAAK KNNVFAWEEV CLTSAYKELF
NKYGINYQQG DIRALLCEQS DKAFYSSFMA LMSLMLQMRN SITGRTDVDF
LISPVKNSDG IFYDSRNYEA QENAILPKNA DANGAYNIAR KVLWAIGQFK
KAEDEKLDKV KIAISNKEWL EYAQTSVK

In certain embodiments, LbCpf1 may comprise an amino acid sequence at least or about 70%, at least or about 75%, at least or about 80%, at least or about 85%, at least or about 90%, at least or about 95%, at least or about 99%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 44.

In certain embodiments, LbCpf1 may comprise an amino acid sequence at least or about 70%, at least or about 75%, at least or about 80%, at least or about 85%, at least or about 90%, at least or about 95%, at least or about 99%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 44, where AsCpf1 contains D833.

In certain embodiments, the effector domain is TET2, Dnmt3b or CTCF. In certain embodiments, the effector domain is CTCF where the polypeptide can modify DNA looping.

The present disclosure provides for a method for modifying an epigenome of a cell. The method may comprise contacting the cell with the present system.

The present disclosure provides for a method for treating a disease in a patient. The method may comprise administering the present system to the patient.

The present polypeptide(s)/system may be used in a method for modifying an epigenome of a cell or a genomic sequence in a cell. The method comprises contacting the cell with the present system/polynucleotide(s). The genomic sequence may be any suitable genomic sequence. In certain embodiments, the genomic sequence may not be, or may be, a BDNF promoter, or may be an enhancer of MyoD.

The present systems/methods may allow precise gene activation or silencing. The present systems/methods may enable multiplex editing of more than one genomic locus. The present systems/methods can allow epigenome editing at multiple sites using a single vector.

U.S. Patent Publication No. 20190359959 is incorporated by reference herein in its entirety.

The present disclosure provides for a method for modifying an X-linked disease-related gene or an imprinting-related disease-related gene in a cell. In certain embodiments, the present systems/methods can be used to treat a disorder/disease. For example, the systems/methods can be applied to reactivate the wild type allele of a gene associated with an X-linked disease selected from Table 1, or a gene associated with an imprinting-related disease selected from Table 2, via epigenetic editing.

The present system may target a target sequence that is associated with a disease-related gene, such as a gene associated with an X-linked disease selected from Table 1, or a gene associated with an imprinting-related disease selected from Table 2.

Table 1 and Table 2 provide an exemplary list of diseases and disease-related genes that can be treated and/or corrected using the present system/method.

In certain embodiments, the disease-related gene is methyl CpG binding protein 2 (MeCP2). MECP2 is a key component of constitutive heterochromatin, which is crucial for chromosome maintenance and transcriptional silencing (Janssen et al., Heterochromatin: guardian of the genome, Annu. Rev. Cell Dev. Biol. 34, 265-288 (2018). Allshire et al., Ten principles of heterochromatin formation and function. Nat. Rev. Mol. Cell Biol. 19, 229-244 (2018). Lyst et al., Rett syndrome: a complex disorder with simple roots. Nat. Rev. Genet. 16, 261-275 (2015)). Mutations in the MECP2 gene cause the progressive neurodevelopmental disorder Rett syndrome (Ip et al., Rett syndrome: insights into genetic, molecular and circuit mechanisms, Nat. Rev. Neurosci. 19, 368-382 (2018). Amir et al., Rett syndrome is caused by mutations in X-linked MECP2, encoding methyl-CpG-binding protein 2, Nat. Genet. 23, 185-188 (1999)), which is associated with severe mental disability and autism-like symptoms that affect girls during early childhood. There are currently no approved treatments for RTT.

TABLE 1
X-linked Diseases

| X-linked disease | Gene | Frequency | Symptoms | Gender |
|---|---|---|---|---|
| Rett Syndrome | MECP2 | 1: 10,000 | Neurological disorder | Mainly |
| (RTT) | (transcription) | | | female; |
| | | | | Male lethal |
| X-linked | PHEX | 1: 20,000 | Increase of FGF23 | Both male |
| hypophosphatemia | (transmembrane | | activity; low level of | and female |
| (XLH)/ | endopeptidase) | | phosphate in the | |
| Hypophosphatemia | | | blood | |
| rickets | | | | |
| Alport | COL4A5 (type IV | 1: 50,000 | Kidney disease, | Het female |
| Syndrome | collagen), 80%; | newborns | hearing loss, and eye | develops |
| | [COL4A3 & | | abnormalities | hematuria |
| | COL4A1 | | | |
| | (autosomal | | | |
| | inheritance) | | | |
| | 15%~20%] | | | |
| Incontinentia | IKBKG | 900-1,200 | Affect the skin, hair, | Mainly |
| pigmenti | (regulator of | affected | teeth, nails and | female; |
| | NF-kB against | individuals | central nervous | Male lethal |
| | apoptosis) | reported | system | |
| Focal dermal | PORCN | Rare disease | Affect the skin, | Male lethal |
| hypoplasia | (palmitoylation of | | skeleton, eyes, and | |
| | Wnt for release) | | face | |
| X-linked dilated | DMD/DYS | Prevalence | Heart disease | Mainly in |
| cardiomyopathy | (Encode | unknown | | males, mild |
| (XLCM) | dystrophin | | | in females |
| *Duchenne | protein) (stabilize | 1: 3,500- | Muscle weakness and | (usually no |
| muscular | muscle fibers) | 5,000 | wasting | symptoms) |
| dystrophy (a | | newborn | | |
| kind of XLCM | | males | | |
| spectrum) | | | | |
| Coffin-Lowry | RPS6KA3 | estimate | Intellectual disability | Both |
| Syndrome (CLS) | (signaling within | 1: 40,000- | and delayed | |
| | cells, control | 50,000 | development | |
| | activity of other | | | |
| | genes) | | | |
| Danon disease | LAMP2 | Rare, exact | Weakening of the | Both; young |
| (glycogen | (lysosomal | prevalence | heart muscle | female may |
| storage disease | associated | unknown | (cardiomyopathy); | have no |
| type IIB, GSD | membrane | | weakening of skeletal | symptom |
| IIB) | protein-2, | | muscles (myopathy); | |
| | transportation) | | and mild intellectual | |
| | | | disability. | |
| Congenital | NSDHL | 60 cases | Affects the | Exclusively |
| hemidysplasia | (production of | reported | development of | in females; |
| with | cholesterol) | | several parts of the | Male lethal |
| ichthyosiform | | | body; typically | |
| erythroderma | | | limited to either | |
| and limb defects | | | right/left side of body | |
| (CHILD | | | | |
| syndrome) | | | | |
| X-linked | PDHA1 (alpha | Unknown | Life-threatening | Normally |
| pyruvate | subunit of | | buildup of lactic acid | male; |
| dehydrogenase | pyruvate | | (lactic acidosis); | female with |
| deficiency | dehydrogenase), | | neurological | skewed X- |
| | more than 80% | | problems; vary | inactivation |
| | | | widely | |
| Cornelia de | HDAC8 (histone | 1: 10,000- | Slow growth, | |
| Lange syndrome | deacetylase 8) or | 30,000 in total | intellectual problem; | |
| | SMC1A (part of | | very widely | |
| | the structural | | | |
| | maintenance of | | | |
| | chromosomes | | | |
| | family), less | | | |
| | common; [if | | | |
| | caused by other 3 | | | |
| | genes, autosomal | | | |
| | inheritance] | | | |
| CDKL5 | CDKL5 (brain | 1: 40,000- | (similar with RTT, | A majority |
| deficiency | development and | 60,000 | previously classified | (more than |
| disorder | function) | | as atypical RTT) | 90%) are |
| | | | Seizure, delay in | girls |
| | | | development | |
| Oral-facial- | OFD1 (may be | 1: 50,000 to | Development of | Predominantly |
| digital syndrome | important for | 250,000 | the oral cavity, facial | female; |
| type I (OFD1) | early | | features, and digits; | Male lethal |
| | development), a | | brain abnormalities; | |
| | majority of OFD | | vary widely | |
| Beta-propeller | WDR45 (encode | Prevalence is | Seizure, intellectual | Most are |
| protein- | WIPI4 protein, | unknown; 35-40% of | disability, et al | female; |
| associated | autophagy) | neurodegeneration | | Male lethal |
| neurodegeneration | | with brain iron | | in most case |
| (BPAN) | | accumulation | | |
| | | (NBIA) disease | | |
| Kabuki | KDM6A (histone | 1: 35,000 | Development delay, | Both |
| syndrome | demethylase), 2-6% | newborns in | intellectual disability; | |
| | | total | eye problem, et al. | |
| CASK-related | CASK (calcium/ | | Intellectual disability | |
| intellectual | calmodulin- | | | |
| disability: two | dependent | | | |
| form | serine protein | | | |
| microcephaly | kinase, regulate | More than 50 | | Most are |
| with pontine and | the movement of | females | | female |
| cerebellar | neurotransmitters | reported | | |
| hypoplasia | and charged | | | |
| (MICPCH) | atoms like ion) | | | |
| X-linked | | More than 20 | | Most are |
| intellectual | | males | | male |
| disability | | reported | | |
| (XL-ID) | | | | |
| X-linked cardiac | FINA | Rare, exact | Vary greatly; Some | |
| valvular | | prevalence | people have no health | |
| dysplasia | | unknown | problems, while in | |
| | | | others blood can leak | |
| | | | through the thickened | |
| | | | and partially closed | |
| | | | valves | |
| X-linked | ALAS2 (5′- | Exact | Vary widely, affect | |
| dominant | aminolevulinate | prevalence | skin, nervous system | |
| protoporphyria | synthase 2 or | unknown | et al | |
| (XLDPP) | erythroid ALA- | | | |
| | synthase, | | | |
| | production of | | | |
| | heme) | | | |
| Mental | HNRNPH2; | | | |
| retardation | MSL3; IQSEC2 | | | |
| (X-linked | | | | |
| dominant) | | | | |

TABLE 2
Imprinting Related Diseases

| Human | | | Mouse | | |
|---|---|---|---|---|---|
| | | Expressed | | | Expressed |
| Gene | Location | allele | Gene | Location | allele |
| NOEY2 | 1p31 | Paternal | | | |
| (ARHI) | | | | | |
| p73 | 1p36 | Maternal | | | |
| U2AFBPL | 5q22-q31 | Biallelic | U2afbp-rs | Proximal 11 | Paternal |
| MAS1 | 6q25.3-q26 | Biallelic/Mono | Mas | Proximal 17 | Paternal |
| | | allelic in breast | | | |
| M6P/IGF2R | 6q26-q27 | Biallelic/Maternal* | M6p/Igf2r | Proximal 17 | Maternal |
| | | | Igf2r-AS | Proximal 17 | Paternal |
| GRB10 | 7p11.2-12 | NR | Meg1/Grb10 | Proximal 11 | Maternal |
| PEG1/MEST | 7q32 | Paternal | Peg1/Mest | Proximal 6 | Paternal |
| WT1 | 11p13 | Biallelic/Maternal* | Wt1 | 2 | NR |
| ASCL2/HASH2 | 11p15.5 | Maternal | Mash2 | Distal 7 | Maternal |
| H19 | 11p15.5 | Maternal | H19 | Distal 7 | Maternal |
| IGF2 | 11p15.5 | Paternal | Igf2 | Distal 7 | Paternal |
| | | | Igf2-AS | Distal 7 | Paternal |
| IMPT1/BWR1A/ | 11p15.5 | Maternal | Impt1 | Distal 7 | Maternal |
| ORCTL2/TSSC5 | | | | | |
| INS | 11p15.5 | Biallelic | Ins2 | Distal 7 | Paternal |
| IPL/TSSC3/ | 11p15.5 | Maternal | Ipl | Distal 7 | Maternal |
| BWR1C | | | | | |
| ITM | 11p15.5 | NR | Itm | Distal 7 | Maternal |
| KvLQT1 | 11p15.5 | Maternal | Kvlqt1 | Distal 7 | Maternal |
| p57^{KIP2}/CDKN1C | 11p15.5 | Maternal | p57^{KIP2} | Distal 7 | Maternal |
| TAPA1 | 11p15.5 | Biallelic^{†} | Tapa1 | Distal 7 | Maternal? |
| HTR2A | 13q14 | Biallelic/Maternal* | Htr2 | 14, Band | Maternal |
| FNZ127 | 15q11-q13 | Paternal | | D3 | |
| GABRA5 | 15q11-q13 | Paternal?^{†} | Gabra5 | Central 7 | Biallelic |
| GABRB3 | 15q11-q13 | Paternal?^{†} | Gabrb3 | Central 7 | Biallelic |
| GABRG3 | 15q11-q13 | Paternal?^{†} | Gabrg3 | Central 7 | Biallelic |
| IPW | 15q11-q13 | Paternal | Ipw | Central 7 | Paternal |
| NDN (necdin) | 15q11-q13 | Paternal | Ndn | Central 7 | Paternal |
| PAR1 | 15q11-q13 | Paternal | | | |
| PAR5 | 15q11-q13 | Paternal | | | |
| PAR-SN | 15q11-q13 | Paternal | | | |
| SNRPN | 15q11-q13 | Paternal | Snrpn | Central 7 | Paternal |
| UBE3A | 15q11-q13 | Maternal | Ube3a | Central 7 | Maternal |
| ZNF127 | 15q11-q13 | Paternal | Zfp127 | Central 7 | Paternal |
| PEG3 | 19q13.4 | Paternal | Peg3/Apoc2 | Proximal 7 | Paternal |
| Neuronatin | 20q11.2-q12 | NR | Peg5/Nnat | Distal 2 | Paternal |
| GNAS1 | 20q13 | Paternal | Gnas1 | Distal 2 | Maternal/Paternal |
| XIST | Xq13.2 | Paternal? | Xist | Xic | Paternal |
| | (XIC)^{‡} | | | | |
| | | | Grf1/Cdc25^{Mm} | Distal 9 | Paternal |
| | | | Impact | Proximal 18 | Paternal |
| | | | Ins1 | Distal 19 | Paternal |

NR, not reported.
*Polymorphic imprinting.
^{†}Determined in vitro.
^{‡}X-inactivation center.
See, Falls et al., Genomic Imprinting: Implications for Human Disease, Am. J. Pathol. 1999; 154(3): 635-647.

In some aspects, one or more nuclear localization sequences (NLS) are fused between the catalytically inactive site specific nuclease (e.g., dCpf1, dCas9, etc.) and the effector domain.

In certain aspects, one or more of the target sequences (e.g., genomic sequences) are associated with a disease or condition.

In certain aspects, the method may further comprise contacting the cell with an agent that inhibits or enhances DNA methylation. The agent may be a small molecule. For example, the agent is 5-azacytidine or 5-azadeoxycytidine.

In certain aspects, the method may further comprise administering to the subject an agent that inhibits or enhances DNA methylation. The agent may be a small molecule. For example, the agent is 5-azacytidine or 5-azadeoxycytidine.

Also disclosed are methods of modulating the expression of one or more genes of interest in a cell, wherein a differentially methylated region is located within 50 kB of the transcription start site of the gene. The method may comprise contacting the cell with the present system, where the guide sequence targets the differentially methylated region.

In some aspects, the differentially methylated region is hypermethylated in the cell and the effector domain (e.g., Tet2 or Tet1) has demethylation activity. In other aspects, the differentially methylated region is unmethylated in the cell and the effector domain (e.g., Dnmt3a) has methylation activity.

The target sequence may comprise a differentially methylated region (DMR). A differentially methylated region may be differentially methylated between cells of different cell types (e.g., muscle cells vs neuron or skin cells vs hepatocytes). A differentially methylated region may be differentially methylated between diseased vs non-diseased cells (e.g., cancer vs non-cancer cells). A differentially methylated region may be differentially methylated between differentiation states (e.g., progenitor cells vs terminally differentiated cells). The effect on expression of one or more genes (e.g., within up to about 0.5, 1, 2, 5, 10, 20, 50, 100, 500 kb or within about 1, 2, 5, or 10 MB from the modification) may be assessed. In some aspects, the differentially methylated region may be hypermethylated or unmethylated.

In some aspects, the present system/method may demethylate a genomic sequence that is aberrantly hypermethylated or may methylate a genomic sequence that is aberrantly unmethylated. In some aspects, an aberrantly hypermethylated sequence or aberrantly unmethylated sequence may occur in a disease or disorder. In other aspects, it is of interest to methylate a CTCF site (e.g., a CTCF binding site) that is aberrantly unmethylated or remove methylation of a CTCF site that is aberrantly methylated. Modifying the methylation or demethylation of the CTCF site may treat or prevent a disease or disorder that exhibits an aberrantly unmethylated sequence or region or an aberrantly hypermethylated sequence or region. For example, a CTCF loop may be opened by methylating a CTCF binding site and thereby bring a gene that is outside the loop under control of an enhancer inside the loop if one wanted to increase expression of that gene (e.g., if expression of the gene is aberrantly low and/or if increased expression is desired for therapeutic or other purposes).

In some aspects, the present system/method may modify a promoter sequence. Targeting of the present system to methylated or unmethylated promoter sequences may cause activation or silencing of expression of a gene.

In some aspects, the present system/method may modify an enhancer sequence. Targeting of the present system to methylated or unmethylated enhancer sequences may cause activation or silencing of expression of a gene.

In some aspects, the present system/method may modify a CTCF binding site. Targeting of the present system to CTCF binding sites may affect CTCF binding and interfere with, or increase, DNA looping, which may alter gene expression (e.g., in the neighboring loop).

In certain embodiments, the guide sequence is an RNA sequence. In one aspect, a single RNA sequence can be complementary to one or more (e.g., all) of the genomic sequences that are being modulated or modified. In one aspect, a single RNA is complementary to a single target genomic sequence. In a particular aspect in which two or more target genomic sequences are to be modulated or modified, multiple (e.g., 2, 3, 4, 5, 6, 7, 8, 9, 10, or more) RNA sequences are used wherein each RNA sequence is complementary to (specific for) one target genomic sequence. In some aspects, two or more, three or more, four or more, five or more, or six or more RNA sequences are complementary to (specific for) different parts of the same target sequence. In one aspect, two or more RNA sequences bind to different sequences of the same region of DNA. In some aspects, a single RNA sequence is complementary to at least two target or more (e.g., all) of the genomic sequences. It will also be apparent to those of skill in the art that the portion of the RNA sequence that is complementary to one or more of the genomic sequences and the portion of the RNA sequence that binds to the catalytically inactive site specific nuclease can be introduced as a single sequence or as 2 (or more) separate sequences into a cell, zygote, embryo or nonhuman animal. In some embodiments, the sequence that binds to the catalytically inactive site specific nuclease comprises a stem-loop.

In certain embodiments, the system contains one or more guide sequences (or a polynucleotide sequence encoding one or more guide sequences) that are complementary to all or a portion of a (one or more) regulatory region, an open reading frame (ORF; a splicing factor), an intronic sequence, a chromosomal region (e.g., telomere, centromere) of the one or more genomic sequences in a cell. In some aspects, the regulatory region targeted by the one or more genomic sequences is a promoter, enhancer, and/or operator region. In some aspects, all or a portion of the regulatory region is targeted by the one or more guide sequences. All or a portion of the region targeted by the one or more guide sequences may be a differentially methylated region. In some aspects, the differentially methylated region is exactly or within about 25 bases, 50 bases, 100 bases, 200 bases, 300 bases, 400 bases, 500 bases, 600 bases, 700 bases, 800 bases, 900 bases, 1000 bases, 1500 bases, 2000 bases, 5000 bases, 10000 bases, 20000 bases, 50000 bases or more upstream to the one or more genes (e.g., endogenous genes; exogenous genes) or a (one or more) transcription start site (TSS). In some aspects, the differentially methylated region is exactly or within about 25 bases, 50 bases, 100 bases, 200 bases, 300 bases, 400 bases, 500 bases, 600 bases, 700 bases, 800 bases, 900 bases, 1000 bases, 1500 bases, 2000 bases, 5000 bases, 10000 bases, 20000 bases, 50000 bases, or more downstream to the one or more genes (e.g., endogenous genes; exogenous genes) or a TSS. The regulatory region targeted by one or more guide sequences may be entirely or partially found at or about the 5′ end of the gene (e.g., endogenous or exogenous) or a TSS. The 5′ end of a gene can include untranscribed (flanking) regions (e.g., all or a portion of a promoter) and a portion of the transcribed region.

As described herein, the one or more guide sequences also comprise a (one or more) binding site for a (one or more) catalytically inactive site specific nuclease. The catalytically inactive site specific nuclease may be a catalytically inactive CRISPR associated (Cas) protein, such as dCpf1. In a particular aspect, upon hybridization of the one or more guide sequences to the one or more target sequences, the catalytically inactive site specific nuclease binds to the one or more guide sequences.

In one aspect, multiple genomic sequences are modulated (e.g., multiplexed activation).

In certain embodiments, the methods further comprise introducing the cell into a non-human mammal. The non-human mammal may be a mouse.

The method may comprise introducing into a cell the present system/polynucleotide(s).

The present disclosure provides for a method of modifying a disease-related gene. The method may comprise introducing into a cell the present system/polynucleotide(s).

In certain embodiments, the guide sequence may comprise a nucleotide sequence at least or about 70%, at least or about 75%, at least or about 80%, at least or about 85%, at least or about 90%, at least or about 95%, at least or about 99%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the nucleotide sequence (or identical to the complementary sequence of the nucleotide sequence) set forth in any of SEQ ID NOs: 14-33.

In certain embodiments, the guide sequence comprises a nucleotide sequence about 80% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleotide sequence (or identical to the complementary sequence of the nucleotide sequence) set forth in any of SEQ ID NOs: 14-33.

The effector domain may have an activity to modify the epigenome of a cell. The effector domain may be a molecule (e.g., protein or a polypeptide) that modulates the expression and/or activation of a genomic sequence (e.g., gene).

In some aspects, the effector domain modifies one or both alleles of a gene. The effector domain can be introduced as a nucleic acid sequence and/or as a protein. In some aspects, the effector domain can be a constitutive or an inducible effector domain. In some aspects, a Cas (e.g., dCpf1) nucleic acid sequence or variant thereof and an effector domain nucleic acid sequence are introduced into the cell as a chimeric sequence. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCpf1) protein or variant thereof and an effector domain are fused or tethered creating a chimeric protein and are introduced into the cell as the chimeric protein. In some aspects, the Cas (e.g., dCpf1) protein and effector domain bind as a protein-protein interaction. In some aspects, the Cas (e.g., dCpf1) protein and effector domain are covalently linked. In some aspects, the effector domain associates non-covalently with the Cas (e.g., dCpf1) protein. In some aspects, a Cas (e.g., dCpf1) nucleic acid sequence and an effector domain nucleic acid sequence are introduced as separate sequences and/or proteins. In some aspects, the Cas (e.g., dCpf1) protein and effector domain are not fused or tethered.

As shown herein, fusions of a catalytically inactive Cas protein (e.g., dCpf1) tethered with all or a portion of (e.g., biologically active portion of) an (one or more) effector domain create chimeric proteins that can be guided to specific DNA sites by one or more guide sequences to modulate activity and/or expression of one or more genomic sequences (e.g., exert certain effects on transcription or chromatin organization, or bring specific kind of molecules into specific DNA loci, or act as sensor of local histone or DNA state). In specific aspects, fusions of dCpf1 tethered with all or a portion of an effector domain create chimeric proteins that can be guided to specific DNA sites by one or more RNA sequences to modulate or modify methylation or demethylation of one or more genomic sequences. As used herein, a “biologically active portion of an effector domain” is a portion that maintains the function (e.g., completely, partially, minimally) of an effector domain (e.g., a “minimal” or “core” domain).

The effector domain may be an enzyme that modifies methylation state of DNA. The effector domain may have methylation activity or demethylation activity (e.g., DNA methylation or DNA demethylation activity). For example, the effector domain may be a DNA methyltransferase (DNMT, such as Dnmt3b and Dmnt3a) or a Ten-Eleven-Translocation (TET) methylcytosine dioxygenase protein (such as Tet2 or Tea). The effector domain may be ACIDA, MBD4, Apobec1, Apobec2, Apobec3, Tdg, Gadd45a, Gadd45b, or ROS1. The effector domain may be Dnmt1, Dnmt3a, Dnmt3b, CpG Methyltransferase M.SssI, or M.EcoHK3 II.

The effector domain may be an enzyme that modifies a histone subunit, such as a histone acetyltransferase (HAT), histone deacetylase (HDAC), histone methyltransferase (HMT), or histone demethylase (e.g., LSD1). In one embodiment, the HAT is p300.

The effector domain may be CTCF, including wild type CTCF or a DNA binding mutant CTCF. In certain embodiments, the DNA binding mutant CTCF comprises one or more of the following mutations: K365A, R368A, R396A, and Q418A.

The effector domain may be a transcriptional activation domain, such as a transcriptional activation domain derived from VP64, VPR or NF-κB p65. The effector domain may be a transcriptional silencer (heterochromatin protein 1 (HP1), or Methyl CpG binding Protein 2 (MeCP2)) or transcriptional repression domain (e.g., a Krueppel-associated box (KRAB) domain, ERF repressor domain (ERD), or mSin3A interaction domain (SID)).

Examples of effector domains also include a transcription(al) activating domain, a coactivator domain, a transcription factor, a transcriptional pause release factor domain, a negative regulator of transcriptional elongation domain, a transcriptional repressor domain, a chromatin organizer domain, a remodeler domain, a histone modifier domain, a DNA modification domain, and a RNA binding domain. Other examples of effector domains include histone marks readers/interactors and DNA modification readers/interactors.

In one aspect of the invention, fusion of the dCpf1 to an effector domain can be to that of a single copy or multiple/tandem copies of full-length or partial-length effector domains. Other fusions can be with split (functionally complementary) versions of the effector domains.

Other examples of effector domains are described in PCT Publication No. WO2014172470 and U.S. Publication No. US20160186208, which are incorporated herein by reference in their entirety.

In some aspects, the Cas (e.g., dCpf1) protein can be fused to the N-terminus or C-terminus of the effector domain.

In one aspect, fusion of dCpf1 with all or a portion of one or more effector domains comprise one or more linkers. In one aspect, a linker comprises one or more amino acids. In some aspects, a linker comprises two or more amino acids. In one aspect, a linker comprises the amino acid sequence GS. In some aspects, fusion of Cas (e.g., dCpf1) with two or more effector domains comprises one or more interspersed linkers (e.g., GS linkers) between the domains. In some aspects, one or more nuclear localization sequences may be located between the catalytically inactive nuclease (e.g., dCpf1) and the effector domain. For example, a fusion protein may include dCpf1-NLS-Tet2, dCpf1-NLS-Dnmt3b, or dCpf1-NLS-CTCF.

In some aspects, one copy of the one or more genomic sequences is modified. In some aspects, both copies of one or more of the genomic sequences in the cell are modified. In some aspects, the one or more genomic sequences that are modified are endogenous to the cell. In particular aspects, at least two of the genomic sequences are endogenous genomic sequences. In some aspects, at least two of the genomic sequences are exogenous genomic sequences. In some aspects where there are at least two genomic sequences, at least one of the genomic sequences is an endogenous genomic sequence and at least one of the genomic sequences is an exogenous genomic sequence. In some aspects, at least two of the genomic sequences are endogenous genes. In some aspects, at least two of the genomic sequences are exogenous genes. In some aspects where there are at least two genomic sequences, at least one of the genomic sequences is an endogenous gene and at least one of the genomic sequences is an exogenous gene. In some aspects, at least two of the genomic sequences are at least 1 kB apart. In some aspects, at least two of the genomic sequences are on different chromosomes.

The present methods may provide for multiplexed epigenome editing in cells. In some aspects, the methods described herein allow for the modification of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc. genomic sequences (e.g., genes) in a (single) cell using the methods described herein. In a particular aspect, one genomic sequence is modified in a (single) cell. In some aspects, two genomic sequences are modified in a (single) cell. In some aspects, three genomic sequences are modified in a (single) cell. In some aspects, four genomic sequences are modified in a (single) cell. In some aspects, five genomic sequences are modified in a (single) cell.

“Modulate” or “modify” means to cause or facilitate a qualitative or quantitative change, alteration, or modification in a level (expression level), an activity, a process, pathway, or phenomenon of interest. Without limitation, such change may be an increase, decrease, or change in relative strength or activity of different components or branches of the process, pathway, or phenomenon.

The present system/method may result in an increase of the expression level or activity of at least one (wildtype) gene or protein, or a decrease of the expression level or activity of at least one (mutant) gene or protein, by at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 91%, at least or about 92%, at least or about 93%, at least or about 94%, at least or about 95%, at least or about 96%, at least or about 97%, at least or about 98%, or at least or about 99%, in about 2 hours, in about 5 hours, in about 10 hours, in about 24 hours, in about 1 day, in about 2 days, in about 3 days, in about 4 days, in about 5 days, in about 6 days, in about 1 week, in about 2 weeks, in about 3 weeks, in about 4 weeks, in about weeks, in about 6 weeks, in about 7 weeks, in about 8 weeks, in about 9 weeks, in about 10 weeks, in about 11 weeks, in about 1 month, in about 2 months, in about 3 months, in about 4 months, in about 5 months, in about 6 months, from about 1 week to about 2 weeks, or within different time-frames following administration to a subject and/or cells (or contacting the cells).

The expression level and/or activity of the (wildtype) gene or protein may increase, or the expression level and/or activity of the (mutant) gene or protein may decrease, by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, at least or about 5%, at least or about 10%, at least or about 15%, at least or about 20%, at least or about 30%, at least or about 40%, at least or about 50%, at least or about 60%, at least or about 70%, at least or about 80%, at least or about 90%, at least or about 100%, about 10% to about 90%, about 12.5% to about 80%, about 20% to about 70%, about 25% to about 60%, or about 25% to about 50%, at least or about 2 fold, at least or about 3 fold, at least or about 4 fold, at least or about 5 fold, at least or about 6 fold, at least or about 7 fold, at least or about 8 fold, at least or about 9 fold, at least or about 10 fold, at least or about 1.5 fold, at least or about 2.5 fold, at least or about 3.5 fold, at least or about 15 fold, at least or about 20 fold, at least or about 50 fold, at least or about 100 fold, at least or about 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 3 fold to about 4 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, compared to a polynucleotide without the target sequence (e.g., the first target sequence), in about 2 hours, in about 5 hours, in about 10 hours, in about 24 hours, in about 1 day, in about 2 days, in about 3 days, in about 4 days, in about 5 days, in about 6 days, in about 1 week, in about 2 weeks, in about 3 weeks, in about 4 weeks, in about 5 weeks, in about 6 weeks, in about 7 weeks, in about 8 weeks, in about 9 weeks, in about 10 weeks, in about 11 weeks, in about 1 month, in about 2 months, in about 3 months, in about 4 months, in about 5 months, in about 6 months, from about 1 week to about 2 weeks, or within different time-frames following administration to a subject and/or cells (or contacting the cells).

The Cas enzyme of the CRISPR/Cas system may be Cas9, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof.

In one embodiment, the Cas enzyme is Cpf1.

As an example, CRISPR/Cas may be encoded by a viral vector, e.g., for therapeutic use.

The gRNA (or crRNA, or sgRNA) may contain a targeting segment that can be fully complementary or substantially complementary (e.g., at least about 70% complementary (e.g., at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target sequence (“target region” or “target DNA”). In certain embodiments, the gRNA (or crRNA, or sgRNA) sequence (or the targeting segment of the gRNA (or crRNA, or sgRNA)) has 100% complementarity to the target sequence. The targeting segment of the gRNA (or crRNA, or sgRNA) may have full complementarity with the target sequence. The targeting segment of the gRNA (or crRNA, or sgRNA) may have partial complementarity with the target sequence. In certain embodiments, the targeting segment of the gRNA (or crRNA, or sgRNA) has or includes 1, 2, 3, 4, 5, 6, 7 or 8 nucleotides that are not complementary with the corresponding nucleotide of the target sequence (mismatches).

In certain embodiments, the gRNA (or crRNA, or sgRNA) is about 10 nucleotides to about 150 nucleotides in length.

In certain embodiments, the targeting segment of the gRNA (or crRNA, or sgRNA) is 16, 17, 18, 19, 20, 21, 22, 23, 24, 25, 26 nucleotides in length. In certain embodiment, the targeting segment of the gRNA (or crRNA, or sgRNA) is 10 to 100, 10 to 90, 10 to 80, 10 to 70, 10 to 60, to 50, 10 to 40, 10 to 30, 10 to 20 or 10 to 15 nucleotides in length. In certain embodiments, the targeting segment of the gRNA (or crRNA, or sgRNA) is 20 to 100, 20 to 90, 20 to 80, 20 to 70, 20 to 60, 20 to 50, 20 to 40, 20 to 30, or 20 to 25 nucleotides in length.

In one embodiment, the degree of complementarity, together with other properties of the gRNA (or crRNA, or sgRNA), is sufficient to allow targeting of a Cas molecule to the target nucleic acid.

In some embodiments, a target sequence is located within an essential gene or a non-essential gene. In an embodiment, the target sequence may be derived from a gene (e.g., a disease-related gene) described herein.

The present disclosure provides a cell comprising: a system described herein, a polypeptide(s) described herein; a nucleic acid(s) described herein; a vector(s) described herein; or a composition described herein.

The cell may be a vertebrate, mammalian (e.g., human), rodent, goat, pig, bird, chicken, turkey, cow, horse, sheep, fish, or primate, cell. The cell may be a plant cell. In an embodiment, the cell is a human cell.

The cell may be somatic cells, stem cells, mitotic or post-mitotic cells, neurons, fibroblasts, or zygotes. A cell, zygote, embryo, or post-natal mammal can be of vertebrate (e.g., mammalian) origin. In some aspects, the vertebrates are mammals or avians. Particular examples include primate (e.g., human), rodent (e.g., mouse, rat), canine, feline, bovine, equine, caprine, porcine, or avian (e.g., chickens, ducks, geese, turkeys) cells, zygotes, embryos, or post-natal mammals. In some embodiments, the cell, zygote, embryo, or post-natal mammal is isolated (e.g., an isolated cell; an isolated zygote; an isolated embryo). In some embodiments, a mouse cell, mouse zygote, mouse embryo, or mouse post-natal mammal is used. In some embodiments, a rat cell, rat zygote, rat embryo, or rat post-natal mammal is used. In some embodiments, a human cell, human zygote or human embryo is used.

The cell may be a somatic cell, germ cell, or prenatal cell. The cell may be a zygotic, blastocyst or embryonic cell, a stem cell, a mitotically competent cell, a meiotically competent cell.

The present system or composition may be introduced into a cell, a zygote, an embryo, a human subject, or a non-human mammal.

In an embodiment, the cell is a cancer cell or other cell characterized by a disease or disorder.

In an embodiment, the target sequence is derived from the nucleic acid of a human cell. In an embodiment, the target sequence is derived from the nucleic acid of: a somatic cell, germ cell, prenatal cell, e.g., zygotic, blastocyst or embryonic, blastocyst cell, a stem cell, a mitotically competent cell, a meiotically competent cell.

In an embodiment, the target sequence is derived from a chromosomal nucleic acid. In an embodiment, the target sequence is derived from an organellar nucleic acid. In an embodiment, the target sequence is derived from a mitochondrial nucleic acid. In an embodiment, the target sequence is derived from a chloroplast nucleic acid.

In an embodiment, the cell is a cell characterized by unwanted proliferation, e.g., a cancer cell. In an embodiment, the cell is a cell characterized by an unwanted genomic component (e.g., a viral genomic component), such as a cell infected with viruses, a cell infected with bacteria etc.

The present disclosure provides a pharmaceutical composition comprising: a polypeptide(s) described herein; a nucleic acid(s) described herein; a vector(s) described herein, a system described herein, or a cell described herein.

The present disclosure provides a method of modulating an epigenome of a cell. The method may comprise contacting the cell with the present polynucleotide(s) (nucleic acid(s)), present system, or present composition.

In an aspect, the disclosure features a method of altering a cell, e.g., altering the structure, e.g., sequence, of a target nucleic acid of a cell, comprising contacting the cell with the present polynucleotide(s) (nucleic acid(s)), present system, or present composition.

In another aspect, the disclosure features a method of treating a subject. The method may comprise administering to the subject (or contacting the cell of the subject), an effective amount of the present polynucleotide(s) (nucleic acid(s)), present system, or present composition.

The present disclosure provides a method of treating a disease or condition in a subject. The method may comprise administering the present polynucleotide(s) (nucleic acid(s)), present composition, present system, or present cells to the subject.

In an embodiment, the subject is an animal or plant. In an embodiment, the subject is a mammalian, primate, or human.

The present disclosure provides a kit comprising: a polypeptide(s) described herein; a nucleic acid(s) described herein; a vector(s) described herein; a system described herein, or a composition described herein. The kit may comprise an instruction for using the system, the polypeptide(s), the nucleic acid(s), the vector(s), or the composition, in a method described herein.

The present system/method may be used to treat a X-linked disease described herein or an imprinting-related disease described herein.

The present disclosure provides for a method for modifying an X-linked disease-related gene or an imprinting-related disease-related gene in a cell. The method may comprise contacting the cell with the present system, polynucleotide(s) or composition.

The cell may be from a subject having a disease, such as an X-linked disease or an imprinting-related disease. The cell may be derived from a cell from a subject having a disease, such as an X-linked disease or an imprinting-related disease.

The cell may be a stem cell, a neuron, a post-mitotic cell, or a fibroblast. In some aspects, the cell is a human cell or a mouse cell.

The cell may be an induced pluripotent stem cell (iPSC), e.g., derived from a fibroblast of a subject. The cell may be an ESC.

The method may further comprise culturing the iPSC or ESC to differentiate into, e.g., a neuron. The method may further comprise administering the differentiated cell (e.g., a neuron) to a subject.

The cell may be autologous or allogeneic to the subject.

The present disclosure provides for a method for treating an X-linked disease or an imprinting-related disease in a subject. The method may comprise administering to the subject a therapeutically effective amount of the present system, polynucleotide(s) or composition.

The terms “disease”, “disorder” or “condition” are used interchangeably and may refer to any alteration from a state of health and/or normal functioning of an organism, e.g., an abnormality of the body or mind that causes pain, discomfort, dysfunction, distress, degeneration, or death to the individual afflicted. Diseases include any disease known to those of ordinary skill in the art. Examples include, e.g., Parkinson's disease, Alzheimer's disease, cancer, hypertension, diabetes mellitus (e.g., type H diabetes mellitus), cardiovascular disease, and stroke (ischemic, hemorrhagic).

In some embodiments, a disease is a psychiatric, neurological, neurodevelopmental disease, neurodegenerative disease, cardiovascular disease, autoimmune disease, cancer, metabolic disease, or respiratory disease. In some embodiments a disease is a psychiatric, neurological, or neurodevelopmental disease, e.g., schizophrenia, depression, bipolar disorder, epilepsy, autism, addiction. Neurodegenerative diseases include, e.g., Alzheimer's disease, Parkinson's disease, amyotrophic lateral sclerosis, frontotemporal dementia.

In some embodiments a disease is an autoimmune diseases e.g., acute disseminated encephalomyelitis, alopecia areata, antiphospholipid syndrome, autoimmune hepatitis, autoimmune myocarditis, autoimmune pancreatitis, autoimmune polyendocrine syndromesautoimmune uveitis, inflammatory bowel disease (Crohn's disease, ulcerative colitis), type I diabetes mellitus (e.g., juvenile onset diabetes), multiple sclerosis, scleroderma, ankylosing spondylitis, sarcoid, pemphigus vulgaris, pemphigoid, psoriasis, myasthenia gravis, systemic lupus erythemotasus, rheumatoid arthritis, juvenile arthritis, psoriatic arthritis, Behcet's syndrome, Reiter's disease, Berger's disease, dermatomyositis, polymyositis, antineutrophil cytoplasmic antibody-associated vasculitides (e.g., granulomatosis with polyangiitis (also known as Wegener's granulomatosis), microscopic polyangjitis, and Churg-Strauss syndrome), scleroderma, Sjogren's syndrome, anti-glomerular basement membrane disease (including Goodpasture's syndrome), dilated cardiomyopathy, primary biliary cirrhosis, thyroiditis (e.g., Hashimoto's thyroiditis, Graves' disease), transverse myelitis, and Guillane-Barre syndrome.

In some embodiments a disease is a respiratory disease, e.g., allergy affecting the respiratory system, asthma, chronic obstructive pulmonary disease, pulmonary hypertension, pulmonary fibrosis, and sarcoidosis.

In some embodiments a disease is a renal disease, e.g., polycystic kidney disease, lupus, nephropathy (nephrosis or nephritis) or glomerulonephritis (of any kind).

In some embodiments a disease is vision loss or hearing loss, e.g., associated with advanced age.

In some embodiments a disease is an infectious disease, e.g., any disease caused by a virus, bacteria, fungus, or parasite.

In some embodiments, a disease exhibits hypermethylation (e.g., aberrant hypermethylation) or unmethylation (e.g., aberrant unmethylation) in a genomic sequence. For example, Fragile X Syndrome exhibits hypermethylation of FMR-1. The present system may be used to specifically demethylate CCG hypermethylation and to reactivate FMG-1, thereby treating Fragile X Syndrome. The methods described herein may be used to treat or prevent diseases or disorders exhibiting aberrant methylation (e.g., hypermethylation or unmethylation).

The polynucleotide/vector may be a recombinant lentiviral vector, or an adeno-associated viral (AAV) vector, such as an AAV2 vector, or an AAV8 vector.

The present system may be delivered by any suitable means. In certain embodiments, the system is delivered in vivo. In other embodiments, the system is delivered to isolated/cultured cells (e.g., autologous iPSC cells) in vitro to provide modified cells useful for in vivo delivery to a subject/patient.

As an alternative to injection of viral particles described in the present disclosure, cell replacement therapy can be used to prevent, correct or treat diseases, where the methods of the present disclosure are applied to isolated patient's cells (ex vivo), which is then followed by the injection of “corrected” cells back into the patient.

In one embodiment, the disclosure provides for introducing the present system or composition into a eukaryotic cell.

The cell may be a stem cell. Examples of stem cells include pluripotent, totipotent, multipotent and unipotent stem cells. Examples of pluripotent stem cells include embryonic stem cells, embryonic germ cells, fetal stem cells, adult stem cells, embryonic carcinoma cells and induced pluripotent stem cells (iPSCs).

The cell may be a somatic cell. Somatic cells may be primary cells (non-immortalized cells), such as those freshly isolated from an animal, or may be derived from a cell line capable of prolonged proliferation in culture (e.g., for longer than 3 months) or indefinite proliferation (immortalized cells). Adult somatic cells may be obtained from individuals, e.g., human subjects, and cultured according to standard cell culture protocols available to those of ordinary skill in the art. Somatic cells of use in aspects of the invention include mammalian cells, such as, for example, human cells, non-human primate cells, or rodent (e.g., mouse, rat) cells. They may be obtained by well-known methods from various organs, e.g., skin, lung, pancreas, liver, stomach, intestine, heart, breast, reproductive organs, muscle, blood, bladder, kidney, urethra and other urinary organs, etc., generally from any organ or tissue containing live somatic cells. Mammalian somatic cells useful in various embodiments include, for example, fibroblasts, Sertoli cells, granulosa cells, neurons, pancreatic cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells, skeletal muscle cells, etc.

For the treatment of a neurological disease, a patient's iPSC cells may be isolated and differentiated into neurons ex vivo. The patient's iPSC cells or neurons characterized by the mutation in a disease-related gene may be manipulated using methods of the present disclosure in a manner that results in the expression of the wildtype allele of a disease-related gene, or the silencing (e.g., transcription being blocked) of a disease-related gene.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by introducing certain factors, referred to as reprogramming factors.

The present methods may further comprise differentiating the iPS cell to a differentiated cell, for example, a neuron.

For example, patient fibroblast cells can be collected from the skin biopsy and transformed into iPS cells. Dimos J T et al. (2008) Induced pluripotent stem cells generated from patients with ALS can be differentiated into motor neurons. Science 321: 1218-1221; Nature Reviews Neurology 4, 582-583 (November 2008). Luo et al., Generation of induced pluripotent stem cells from skin fibroblasts of a patient with olivopontocerebellar atrophy, Tohoku J. Exp. Med. 2012, 226(2): 151-9. The CRISPR-mediated modification can be done at this stage. The corrected cell clone can be screened and selected by RFLP assay. The corrected cell clone is then differentiated into, e.g., neurons and tested for its neuron-specific markers. Well-differentiated neurons can be transplanted autologously back to the donor patient.

The cell may be autologous or allogeneic to the subject who is administered the cell.

The term “autologous” refers to any material derived from the same individual to whom it is later to be re-introduced into the same individual.

The term “allogeneic” refers to any material derived from a different animal of the same species as the individual to whom the material is introduced. Two or more individuals of the same species are said to be allogeneic to one another.

The corrected cells for cell therapy to be administered to a subject. Cells (e.g., neurons) described in the present disclosure may be formulated with a pharmaceutically acceptable carrier. For example, cells can be administered alone or as a component of a pharmaceutical formulation. The cells (e.g., neurons) can be administered in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

Subjects, which may be treated according to the present disclosure, include all animals which may benefit from the present invention. Such subjects include mammals, preferably humans (infants, children, adolescents and/or adults), but can also be an animal such as dogs and cats, farm animals such as cows, pigs, sheep, horses, goats and the like, and laboratory animals (e.g., rats, mice, guinea pigs, and the like).

The terms “polynucleotide”, “nucleotide”, “nucleotide sequence”, “nucleic acid” and “oligonucleotide” are used interchangeably. These terms refer to a polymeric form of nucleotides of any length, either deoxyribonucleotides or ribonucleotides, or analogs. Examples of polynucleotides include, but are not limited to, DNA, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide sequence can further be modified. The sequence of nucleotides may be interrupted by non-nucleotide components. A polynucleotide may also be modified after polymerization, such as by conjugation with a labeling agent.

The term “Cas9” refers to a CRISPR associated endonuclease referred to by this name. Non-limiting exemplary Cas9s are provided herein, e.g. the Cas9 provided for in UniProtKB G3ECR1 (CAS9 STRTR) or the Staphylococcus aureus Cas9, as well as the nuclease dead Cas9, orthologs and biological equivalents each thereof. Orthologs include but are not limited to Streptococcus pyogenes Cas9 (“spCas9”); Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA may comprise, or alternatively consist essentially of, or yet further consist of, a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas or equivalent thereof to a specific nucleotide sequence such as a specific region of a cell's genome.

A nuclease-defective or nuclease-deficient Cas protein (e.g., dCas9) with one or more mutations on its nuclease domains retains DNA binding activity when complexed with a guide sequence (e.g., gRNA). dCas protein can tether and localize effector domains or protein tags by means of protein fusions to sites matched by gRNA, thus constituting an RNA-guided DNA binding enzyme.

gRNAs can be generated to target a specific gene, optionally a gene associated with a disease, disorder, or condition. Thus, in combination with Cas, the guide RNAs facilitate the target specificity of the CRISPR/Cas system. Further aspects such as promoter choice, as discussed herein, may provide additional mechanisms of achieving target specificity—e.g., selecting a promoter for the guide RNA encoding polynucleotide that facilitates expression in a particular organ or tissue. Accordingly, the selection of suitable gRNAs for the particular disease, disorder, or condition is contemplated herein.

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. As a non-limiting example, in some cases, the dCas9 harbors both D10A and H840A mutations of the amino acid sequence of S. pyogenes Cas9. In some embodiments when a dCas9 has reduced or defective catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

The present disclosure provides for gene editing methods that can modify the disease-related gene, which in turn can be used for in vivo gene therapy for patients afflicted with the disease.

The nuclease (e.g., dCpf1) can be introduced into the cell in the form of a DNA, mRNA or protein. The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics.

The guide sequence (e.g., crRNA, sgRNA, gRNA, etc.) used in the present system/method can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, the guide sequence (e.g., crRNA, sgRNA, gRNA, etc.) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

The methods of the present disclosure can also be used to prevent, correct, or treat cancers that arise due to the presence of mutation in a tumor suppressor gene. Examples of tumor suppression genes include, retinoblastoma susceptibility gene (RB) gene, p53 gene, deleted in colon carcinoma (DCC) gene, adenomatous polyposis coli (APC) gene, p16, BRCA1, BRCA2, MSH2, and the neurofibromatosis type 1 (NF-1) tumor suppressor gene (Lee at al. Cold Spring Harb Perspect Biol. 2010 October; 2(10)).

The methods of the present disclosure may be used to treat patients at a different stage of the disease (e.g., early, middle or late). The present methods may be used to treat a patient once or multiple times. Thus, the length of treatment may vary and may include multiple treatments.

Furthermore, methods of the present disclosure may be applied to specific gene-humanized mouse model as well as patient-derived cells, allowing for determining the efficiency and efficacy of designed sgRNA and site-specific recombination frequency in human cells, which can be then used as a guide in a clinical setting.

A variety of viral constructs may be used to deliver the present system to the targeted cells and/or a subject. Non-limiting examples of such recombinant viruses include recombinant lentiviruses, recombinant adeno-associated virus (AAV), recombinant adenoviruses, recombinant retroviruses, recombinant poxviruses, and other known viruses in the art, as well as plasmids, cosmids, and phages. Options for gene delivery viral constructs are well known (see, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71).

AAV viral vectors may be selected from among any AAV serotype, including, without limitation, AAV1, AAV2, AAV3, AAV4, AAV5, AAV6, AAV7, AAV8, AAV9, AAV10 or other known and unknown AAV serotypes. In certain embodiment, AAV2 and/or AAV8 are used.

The term AAV covers all subtypes, serotypes and pseudotypes, and both naturally occurring and recombinant forms, except where required otherwise. Pseudotyped AAV refers to an AAV that contains capsid proteins from one serotype and a viral genome of a second serotype.

Additionally, delivery vehicles such as nanoparticle- and lipid-based mRNA or protein delivery systems can be used as an alternative to viral vectors. Further examples of alternative delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83).

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

The recombinant viral vector(s) containing the desired recombinant DNA can be formulated into a pharmaceutical composition. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution. In another embodiment, the carrier is balanced salt solution. In one embodiment, the carrier includes Tween. If the virus is to be stored long-term, it may be frozen in the presence of glycerol or Tween-20.

The present system, cells or compositions may be administered by, direct delivery to a desired organ or tissue, injection, oral, inhalation, intranasal, intratracheal, intravenous, intramuscular, subcutaneous, intradermal, and other parental routes of administration. Additionally, routes of administration may be combined, if desired. Administration may be through any suitable routes, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art.

The term “about,” as used herein when referring to a numerical value, is meant to encompass variations of 20%, 10%, 5%, 1%, 0.5%, or 0.1% of the specified amount.

As used herein, “treating” or “treatment” of a disease or a condition in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention.

The following examples of specific aspects for carrying out the present invention are offered for illustrative purposes only, and are not intended to limit the scope of the present invention in any way.

### Example 1 Multiplex Epigenome Editing Using dCpf1

We tested a series of engineered chimeric proteins in which dCpf1 was fused with effector proteins such as p300 to mediate targeted histone acetylation, or CTCF to mediate targeted DNA looping. We validated these epigenome editing tools in manipulating gene expression and 3D chromatin structures.

Cpf1 is sufficient to generate several crRNAs from a single transcript (designed CRISPR array) to target multiple sequences. An “all-in-one” vector (e.g., a plasmid) encoding a crRNA array, Cpf1, and a selection marker may be used in the present method (FIG. 1).

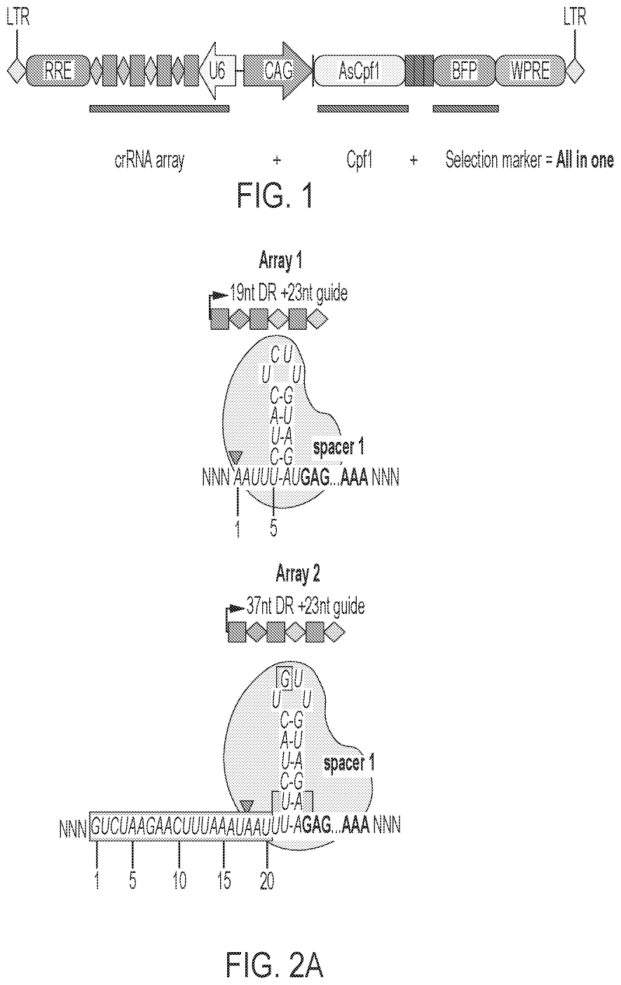

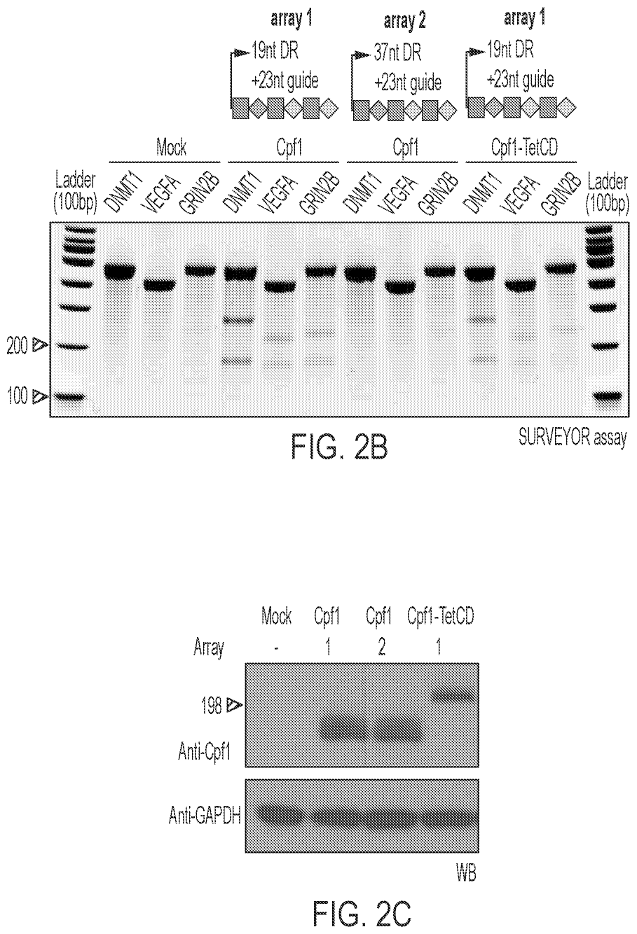

The ability of Cpf1 with different arrays to induce indels at the DNMT1, VEGFA, GRIN2B targets were examined by the Surveyor assay (FIG. 2B). Array 1 contained 19 nucleotide (nt) DR and 23 nt guide RNA (gRNA), while Array 2 had 37 nt DR and 23 nt gRNA.

We used HEK293T cells to test AsCpf1 with different direct repeats (DR). After each construct plasmid was transfected into HEK293T cells, genomic DNA was extracted for the Surveyor assay to compare the cutting efficiencies on the DNMT1, VEGFA, and GRIN2B loci with the target sequences listed below. Our result showed that 19 nt DR (UAAUUUCUACUCUUGUAGAU; SEQ ID NO: 1) worked better than the 37 nt DR (FIG. 2B). The expression of each construct was validated by Western blot (FIG. 2C).

Target sequences:
DNMT1:
(SEQ ID NO: 2)
TTAATGTTTCCTGATGGTCCATGTCTGTTACTCGCCTGTCAA
VEGFA:
(SEQ ID NO: 3)
TCCCTCTTTGCTAGGAATATTGAAGGGGGCAGGGGAAGGCGG
GRIN2b:
(SEQ ID NO: 4)
GTTGGGTTTGGTGCTCAATGAAAGGAGATAAGGTCCTTGAAT

The results show that Cpf1 has multiplex targeting ability, and that the DR sequence in Array 2 was not as effective as Array 1. Additionally, the Cpf1-TetCD fusion protein maintained both the Cpf1 RNase and DNase activities.

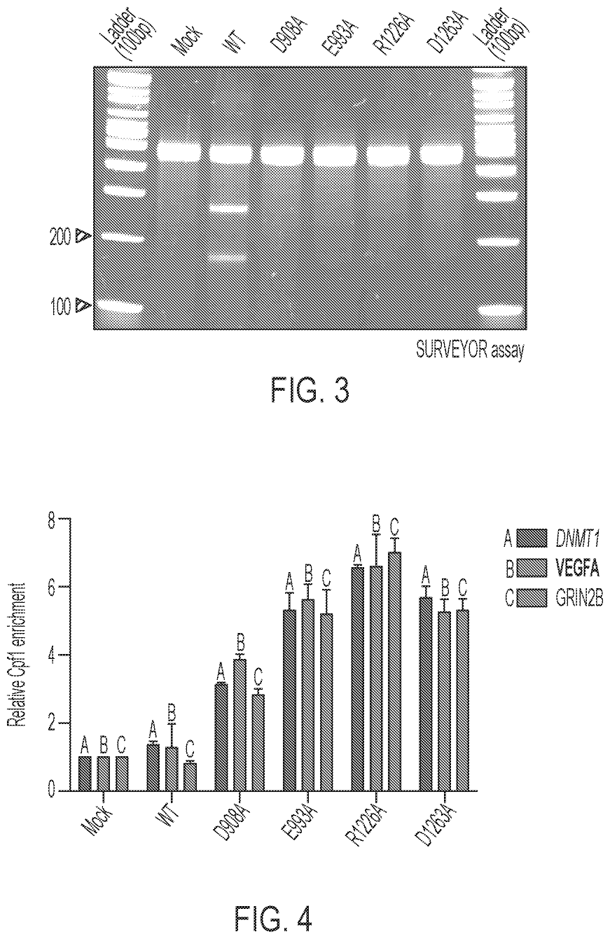

We used HEK293T cells to test which point mutations abolished the Dnase activity of AsCpf1. After each construct plasmid was transfected into HEK293T cells, genomic DNA was extracted for the Surveyor assay to compare the cutting efficiencies on the DNMT1 locus with the target sequence listed above (SEQ ID NO: 2). The results in FIG. 3 show that the point mutations D908A, E993A, R1226A and D1263A in the RuvC and NuC domains silenced the AsCpf1 DNase activity (DNase activity catalytically dead Cpf1).

Affinity analysis of key residues in the RuvC and Nuc domains of AsCpf1 was conducted. Effects of point mutations on the ability of AsCpf1 (DNase activity catalytically dead Cpf1) to bind to the DNMT1, VEGFA and GRIN2B target DNA sequences were examined using chromatin immunoprecipitation (ChIP)-qPCR (n=3, error bars show mean±SEM). Values were normalized against the mock sample. The results in FIG. 4 show that mutation R1226A presented the highest affinity towards the DNA targets.

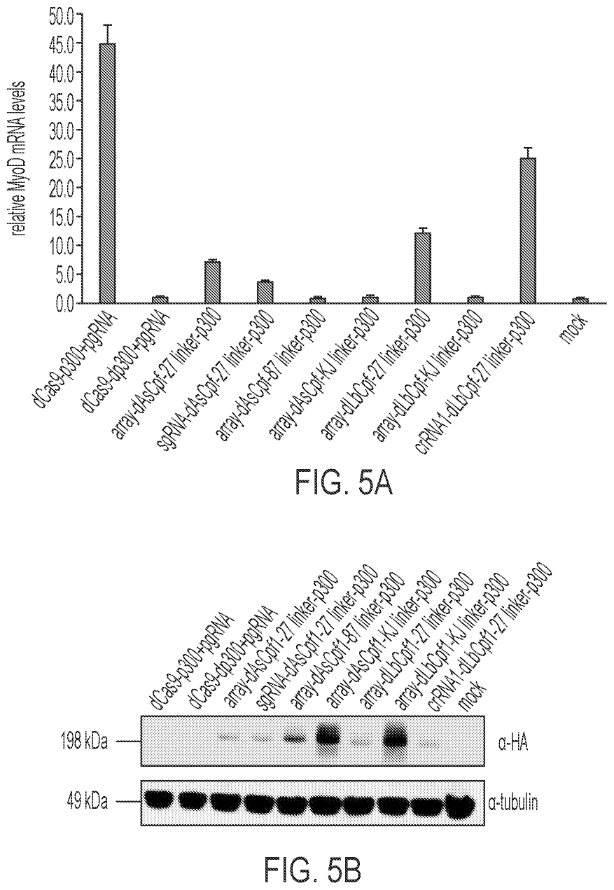

We used HEK293T cells to test which orthologue(s) of Cpf1 can be used to fuse with p300 to mediate target histone acetylation for gene activation. After each construct plasmid was transfected into HEK293T cells, RNA was extracted to perform qPCR to compare the expressions of targeted MyoD locus. dCas9 is Cas9 with the following point mutations: D10A and H840A; dAsCpf1 is AsCpf1 with the following point mutations: D908A, E993A, R1226A and D1263A; dLbCpf1 is LbCpf1 with the following point mutation: D833A. Our result (FIGS. 5A-5B) showed that catalytically dead LbCpf1 with a 27 amino acid linker worked the best to activate MyoD mRNA expression compared to dCas9-p300. The amino acid sequence of the 27 amino acid linker is: GGGGSPKKKRKVGPKKKRKVDGGGGSE (SEQ ID NO: 7). The nucleotide sequence encoding the 27 amino acid linker is:

(SEQ ID NO: 8)
ggtggcggaggctcgccaaaaaagaagagaaaggtaggtccaaagaaaa
aacgaaaagtagatggtggcggaggatccgaa.

The target sequence of MyoD is listed below.

CX_ANL083-Cpf1-MyoD-g1(23 nt) (promoter):
(SEQ ID NO: 9)
taaaaaaaTTGGCTCTCCGGCACGCCCTTTCATCTACAAGAGTAGAAAT
TGACG
CX_ANL084-Cpf1-MyoD-g1(23 nt) (promoter):
(SEQ ID NO: 10)
CTAGCGTCAATTTCTACTCTTGTAGATGAAAGGGCGTGCCGGAGAGCCA
Atttttttaat

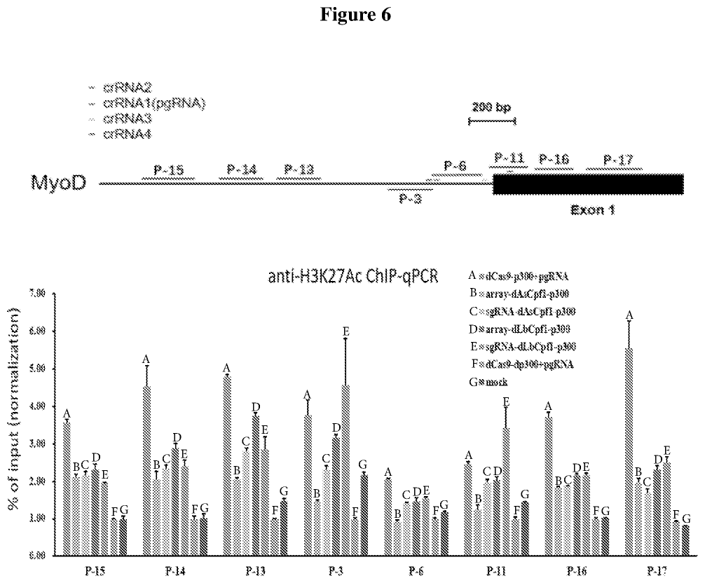

The effective range of dLbCpf1-p300 was also studied. After each construct plasmid was transfected into HEK293T cells, ChIP-qPCR using anti-H3K27Ac antibody was performed to compare the acetylation levels in the targeted MyoD locus. Our results in FIG. 6 showed that the effective range of dLbCpf1-p300 is about 2000 bp upstream of the crRNA and about 1000 bp downstream of the crRNA. dCas9 is Cas9 with the following point mutations: D10A and H840A; dAsCpf1 is AsCpf1 with the following point mutations: D908A, E993A, R1226A and D1263A; dLbCpf1 is LbCpf1 with the following point mutation: D833A.

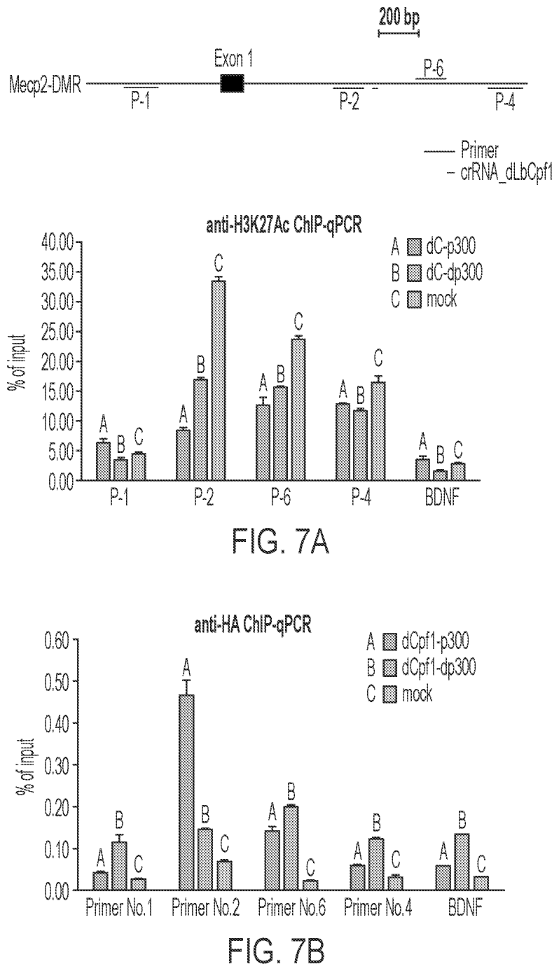

FIGS. 7A-7B shows the results to study the effective range of editing H3K27 acetylation at the MeCP2 locus by the dCpf1-p300 system. In FIG. 7A, anti-H3K27Ac antibody was used for ChIP-qPCR. In FIG. 7B, anti-HA antibody was used for ChIP-qPCR. dLbCpf1 or dCpf1 is LbCpf1 with the following point mutation: D833A.

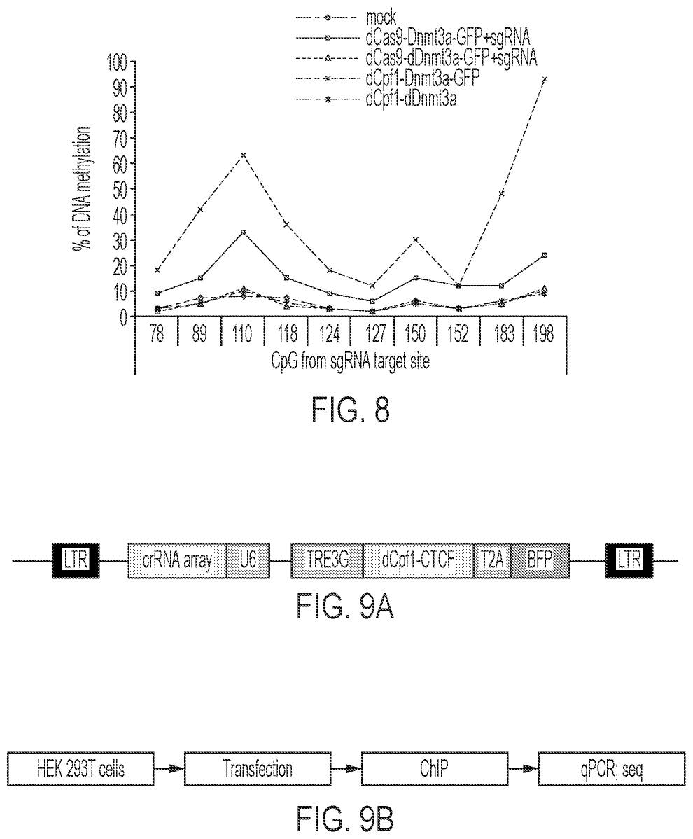

FIG. 8 shows that dCpf1-Dnmt3a provides higher DNA methylation editing efficiency than dCas9-Dnmt3. dCas9 is Cas9 with the following point mutations: D10A and H840A; dCpf1 is LbCpf1 with the following point mutation: D833A.

sgRNA was designed to target the p16 locus (SEQ
ID NO: 11):
atttggcagttaggaaggttgtatcgcggaggaaggaaacggggcgggg
gcggatttctttttaacagagtgaacgcactcaaacacgcctttgctgg
caggcgggggagcgcggctgggagcagggaggccggagggcggtgtggg
gggcaggtggggaggagcccagtcctccttccttgccaacgctggctct
ggcgagggctgcttccggctggtgcccccgggggagacccaacctgggg
cgacttcaggggtgccacattcgctaagtgctcggagttaatagcacct
cctccgagcactcgctcacggcgtccccttgcctggaaagataccgcgg
tccctccagaggatttgagggacagggtcggagggggctcttccgccag
caccggaggaagaaagaggaggggctggctggtcaccagagggtggggc
ggaccgcgtgcgctcggcggctgcggagagggggagagcaggcagcggg
ggggggagcagcATGGAGCCGGCGGCGGGGAGCAGCATGGAGCCTTCGG
CTGACTGGCTGGCCACGGCCGCGGCCCGGGGTCGGGTAGAGGAGGTGCG
GGCGCTGCTGGAGGCGGGGGCGCTGCCCAACGCACCGAATAGTTACGGT
CGGAGGCCGATCCAGGTGGGTAGAGGGTCTGCAGCGGGAGCAGGGGATG
GCGGGCGACTCTGGAGGACGAAGTTTGCAGGGGAATTGGAATCAGGTAG
CGCTTCGATTCTCCGGAAAAAGGGGAGGCTTCCTGG

The sgRNA sequences are tcctccttccttgccaacgctggct (SEQ ID NO: 12; used with dCas9-Dnmt3a) and gctggcaggcgggggagcgcgg (SEQ ID NO: 13; used with dCpf1-Dnmt3a).

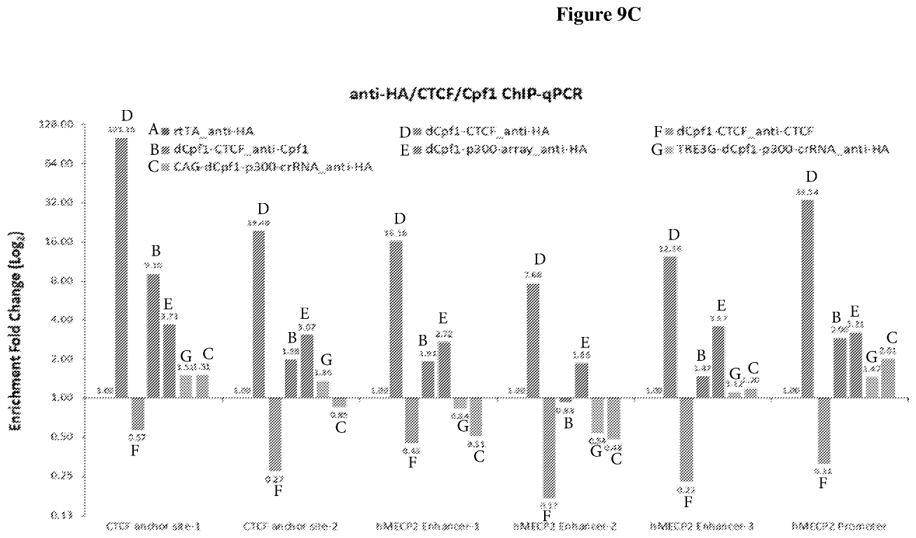

We tested whether dCpf1-CTCF can be targeted to multiple CTCF anchor sites. After each construct plasmid was transfected into HEK293T cells, ChIP-qPCR using antibodies against Cpf1-HA or CTCF was performed to examine the binding of dCpf1-CTCF or dCpf1-p300 to the targeted MeCP2 locus. Our results (FIGS. 9A-9C) showed that dCpf1-CTCF can be detected at the targeted genomic sites. dCpf1 is LbCpf1 with the following point mutation: D833A.

It was reported that the mutations of certain CTCF amino acid residues can reduce the affinity between CTCF and DNA. The CTCF mutants include CTCF(K365A), CTCF(R368A), CTCF(K365A, R368A), CTCF(R396A) and CTCF(Q418A) (Yin et al., Molecular mechanism of directional CTCF recognition of a diverse range of genomic sites, Cell Research (2017):1365-1377).

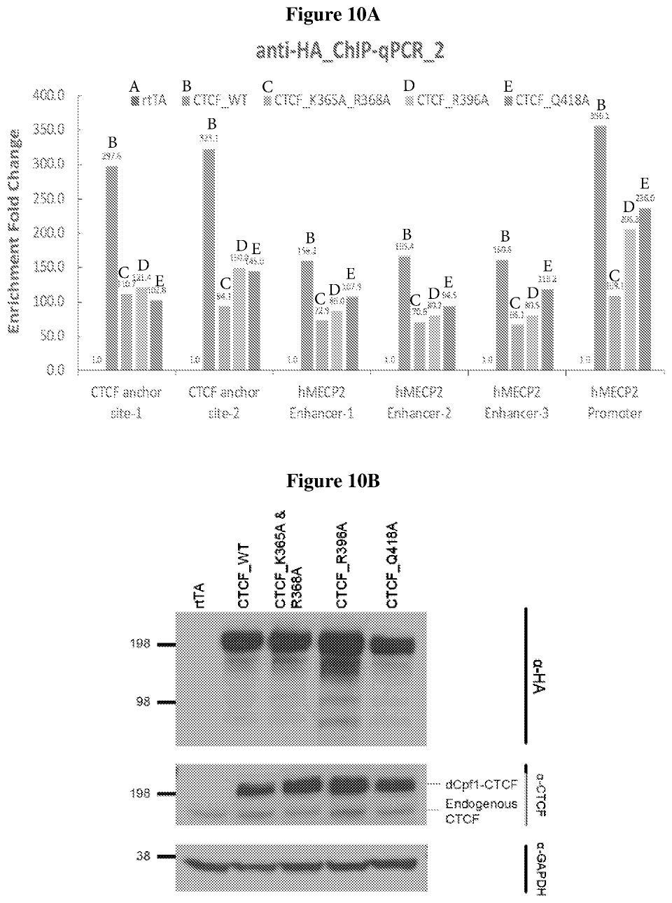

DNA-binding mutants of CTCF reduced the off-target effect of dCpf1-CTCF (FIGS. 10A-10B). ChIP-qPCR was performed using anti-HA antibodies to examine the binding of dCpf1-CTCF to the targeted MeCP2 locus (FIG. 10A). dCpf1 is LbCpf1 with the following point mutation: D833A.

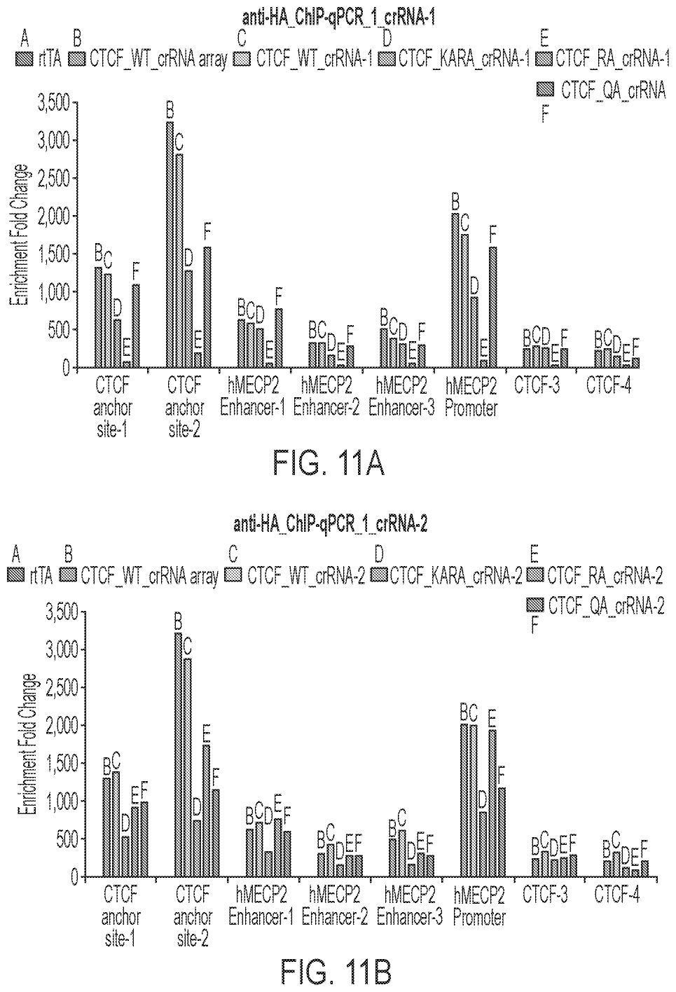

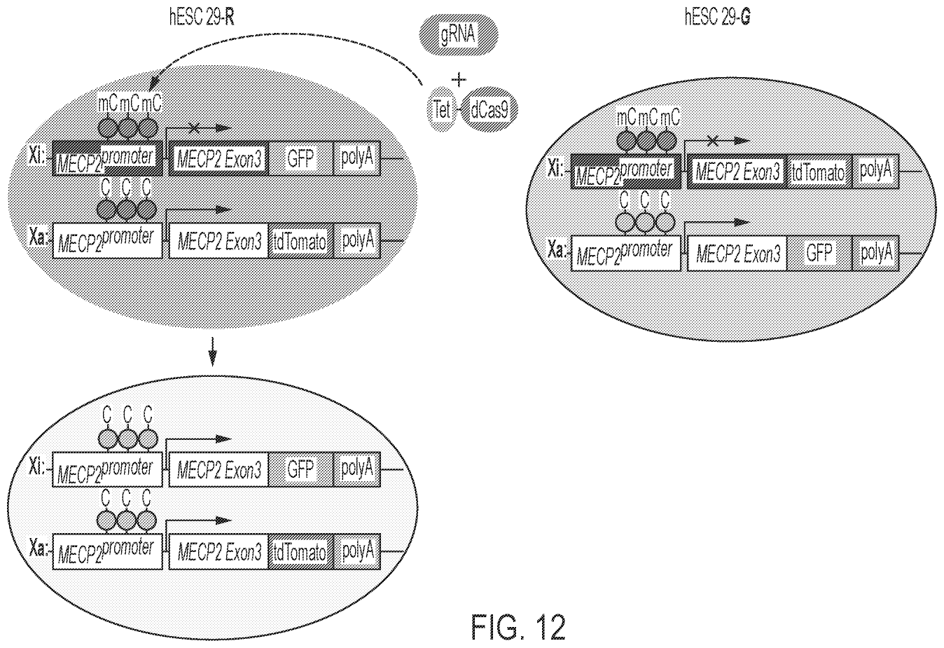

FIGS. 11A-11B show dCpf1-CTCF mediated DNA looping/binding of the MeCP2 locus using either crRNA-1 (FIG. 11A) or crRNA-2 (FIG. 11B).

### Example 2 Multiplex Epigenome Editing Reactivates MeCP2 to Rescue Rett Syndrome Neurons

Rett syndrome is a neurological disorder mainly observed in girls (1 in 8,500). The symptoms include smaller brain size (microcephaly), inability to speak, loss of purposeful use of the hands, problems with walking, and abnormal breathing pattern.

Rett syndrome is caused by heterozygous mutation of MECP2 on the X chromosome. We applied the newly developed tool (including dCas9-Tet and dCpf1-CTCF) to reactivate the wild-type allele of the MECP2 gene on the inactive X chromosome as a therapeutic strategy for Rett syndrome. We used Rett syndrome-like hESCs and neurons derived from this hESC line, and performed multiplex epigenome editing.

The results show that we can specifically reactivate the MECP2 allele on the inactive X chromosome in Rett syndrome-like hESCs and derive functionally rescued neurons. We can also combine dCas9-Tet-mediated DNA methylation editing with dCpf1-CTCF-mediated DNA looping to achieve stable reactivation of the wildtype MECP2 allele on the inactive X chromosome in neurons. The present system/method may also be used to treat other X-linked diseases.

MECP2 dual color reporter (FIG. 12) allows: 1) detection of MECP2 reactivation on Xi; 2) examining the editing effect on Xa; and 3) assessing off-target effects.

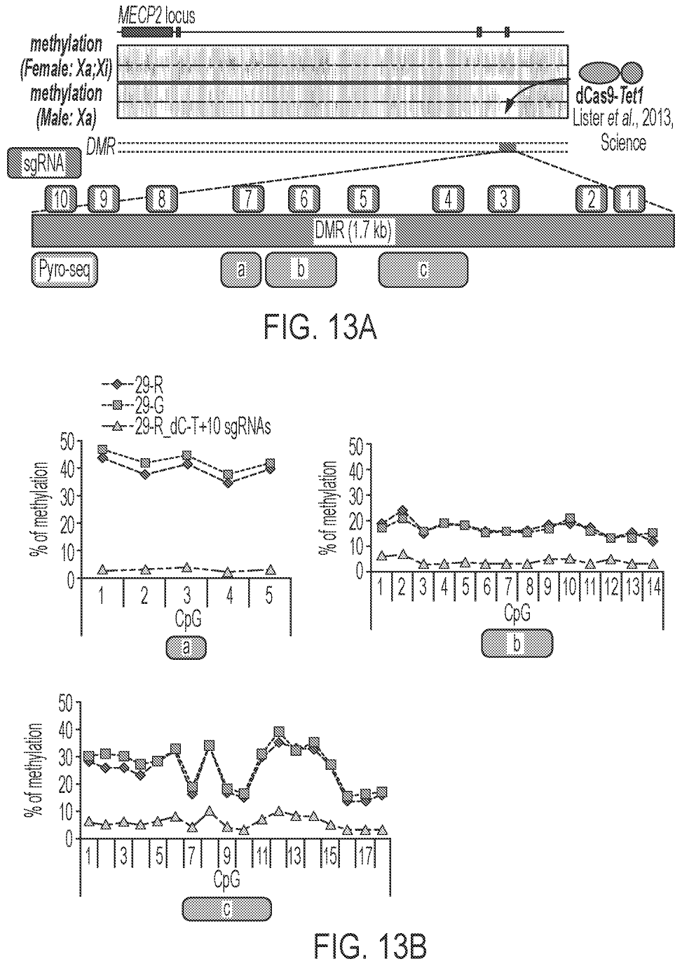

Demethylation of the Xi-specific DMR at the MECP2 promoter by dCas9-Tet1 was studied (FIGS. 13A-13B). FIG. 13A is a schematic representation of the MECP2 promoter (Lister et al., Global Epigenomic Reconfiguration During Mammalian Brain Development, Science, 2013, 341(6146):1237905) targeted by sgRNAs including sgRNA-1 to sgRNA-10, as well as the regions (Regions a-c) for pyrosequencing (pyro-seq). FIG. 13B shows the pyrosequencing (pyro-seq) results for Regions a-c. dCas9 is Cas9 with the following point mutations: D10A and H840A. sgRNAs including sgRNA-1 to sgRNA-10 targeting the DMR in human MeCP2 promoter region are as follows.

SL-586_hMeCP2_DMR_sgRNA-1_For:
(SEQ ID NO: 14)
TTGG AGCAGCAAAGTTGCCCACCC
SL-587_hMeCP2_DMR_sgRNA-1_Rev:
(SEQ ID NO: 15)
AAAC GGGTGGGCAACTTTGCTGCT
SL-588_hMeCP2_DMR_sgRNA-2_For:
(SEQ ID NO: 16)
TTGG TAGTGATATTGAGAAAATGT
SL-589_hMeCP2_DMR_sgRNA-2_Rev:
(SEQ ID NO: 17)
AAAC ACATTTTCTCAATATCACTA
SL-590_hMeCP2_DMR_sgRNA-3_For:
(SEQ ID NO: 18)
TTGG CAGCCAATCAACAGCTGGAG
SL-591_hMeCP2_DMR_sgRNA-3_Rev:
(SEQ ID NO: 19)
AAAC CTCCAGCTGTTGATTGGCTG
SL-592_hMeCP2_DMR_sgRNA-4_For:
(SEQ ID NO: 20)
TTGG GCCATCACAGCCAATGAC
SL-593_hMeCP2_DMR_sgRNA-4_Rev:
(SEQ ID NO: 21)
AAAC GTCATTGGCTGTGATGGC
SL-594_hMeCP2_DMR_sgRNA-5_For:
(SEQ ID NO: 22)
TTGG AGGAGGAGAGACTGTGAGT
SL-595_hMeCP2_DMR_sgRNA-5_Rev:
(SEQ ID NO: 23)
AAAC ACTCACAGTCTCTCCTCCT
SL-596_hMeCP2_DMR_sgRNA-6_For:
(SEQ ID NO: 24)
TTGG GGAGGGGGAGGGTAGAGAGG
SL-597_hMeCP2_DMR_sgRNA-6_Rev:
(SEQ ID NO: 25)
AAAC CCTCTCTACCCTCCCCCTCC
SL-598_hMeCP2_DMR_sgRNA-7_For:
(SEQ ID NO: 26)
TTGG GGGAGGAAGAGGGGCGTC
SL-599_hMeCP2_DMR_sgRNA-7_Rev:
(SEQ ID NO: 27)
AAAC GACGCCCCTCTTCCTCCC
SL-600_hMeCP2_DMR_sgRNA-8_For:
(SEQ ID NO: 28)
TTGG TGAGAGCTCAGGAGCCCTTG
SL-601_hMeCP2_DMR_sgRNA-8_Rev:
(SEQ ID NO: 29)
AAAC CAAGGGCTCCTGAGCTCTCA
SL-602_hMeCP2_DMR_sgRNA-9_For:
(SEQ ID NO: 30)
TTGG CCTACTTGTTCCTGCTAGAT
SL-603_hMeCP2_DMR_sgRNA-9_Rev:
(SEQ ID NO: 31)
AAAC ATCTAGCAGGAACAAGTAGG
SL-604_hMeCP2_DMR_sgRNA-10_For:
(SEQ ID NO: 32)
TTGG AGGTGGTTATAGTTCCCATC
SL-605_hMeCP2_DMR_sgRNA-10_Rev:
(SEQ ID NO: 33)
AAAC GATGGGAACTATAACCACCT

For pyro-seq of the hMECP2 promoter, Region a was amplified with the following primers and sequenced by the sequencing primer accordingly.

SL-813_hMECP2 promoter_No1_For:
(SEQ ID NO: 34)
GAGGGGGAGGGTAGAGAG
SL-814_hMECP2 promoter_No1_Rev_Biotin:
(SEQ ID NO: 35)
CTCCCTCCTCTCCAAAAAAAAACTATAATA
SL-815_hMECP2 promoter_No1_Seq:
(SEQ ID NO: 36)
GGGAGGGTAGAGAGG

Region b was amplified with the following primers and sequenced by the sequencing primer accordingly.

SL-816_hMECP2 promoter_No2_For:
(SEQ ID NO: 37)
GGGTAGAGGGGGGTAGAAATT
SL-817_hMECP2 promoter_No2_Rev_Biotin:
(SEQ ID NO: 38)
ACCCCCACCTCTCCCTAAAT
SL-818_hMECP2 promoter_No2_Seq:
(SEQ ID NO: 39)
AGAGTTTAGGAGTTTTTGT

Region c was amplified with the following primers and sequenced by the sequencing primer accordingly.

SL-819_hMECP2 promoter_No3_For:
(SEQ ID NO: 40)
GAGTTGTGGGATTTAGAATATAATGT
SL-820_hMECP2 promoter_No3_Rev_Biotin:
(SEQ ID NO: 41)
CTCCTTCTCCCCCATTCCATAAATTTC
SL-821_hMECP2 promoter_No3_Seq:
(SEQ ID NO: 42)
GTTAGATGGGGAAAGG

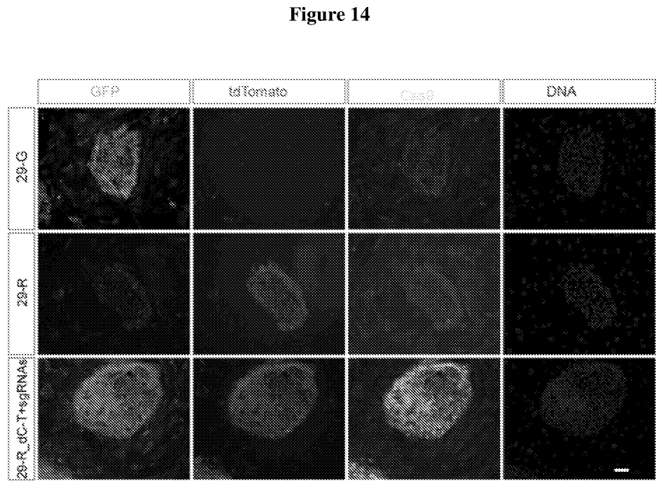

Cells were infected with lentiviruses expressing dCas9-Tet1-P2A-BFP (dC-T) and lentiviruses expressing sgRNA-mCherry (10 sgRNAs as discussed above were used). Fluorescence-activated cell sorting (FACS) was used to isolate cells that were BFP+mCherry+. Infected cells were subject to immunofluorescence staining. The immunofluorescence images suggested that methylation editing resulted in reactivation of MECP2 on the inactive X chromosome (Xi) in hESCs (FIG. 14). dC-T: dCas9-Tet1. dCas9 is Cas9 with the following point mutations: D10A and H840A.

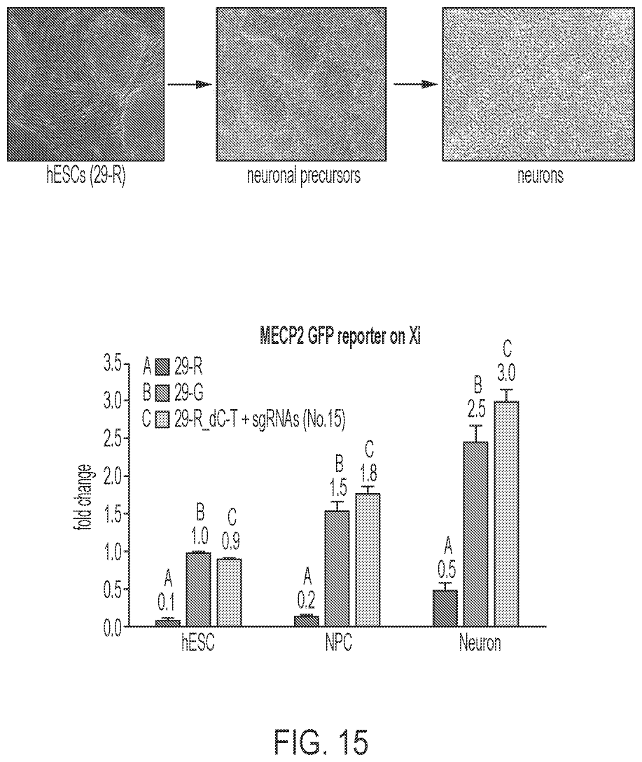

MECP2 reactivation was maintained in neural precursor cells (NPCs) and neurons (FIG. 15). dCas9 is Cas9 with the following point mutations: D10A and H840A.

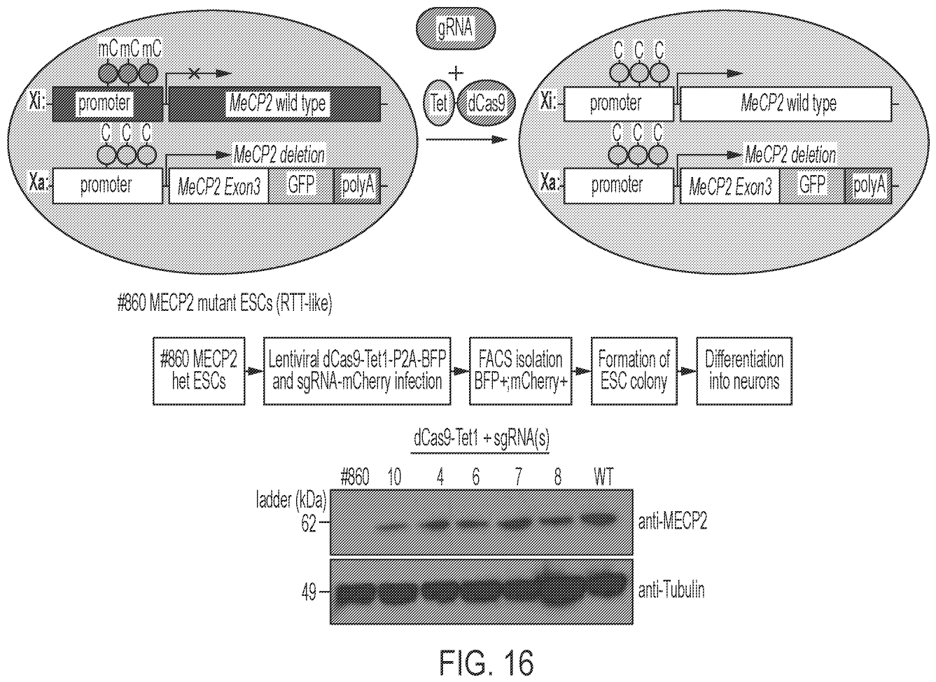

MECP2 mutant #860 RTT-like human embryonic stem cells (hESC) were infected with lentiviruses expressing dCas9-Tet1-P2A-BFP (dCas9-Tet1) and lentiviruses expressing sgRNA-mCherry (10 sgRNAs). Fluorescence-activated cell sorting (FACS) was used to isolate cells that were BFP+mCherry+, which were cultured to form ESC colonies. The ESCs were then allowed to differentiate into neurons. The results show that dCas9-Tet1 in combination with a single sgRNA was sufficient to reactivate MECP2 on Xi (FIG. 16). dCas9 is Cas9 with the following point mutations: D10A and H840A.

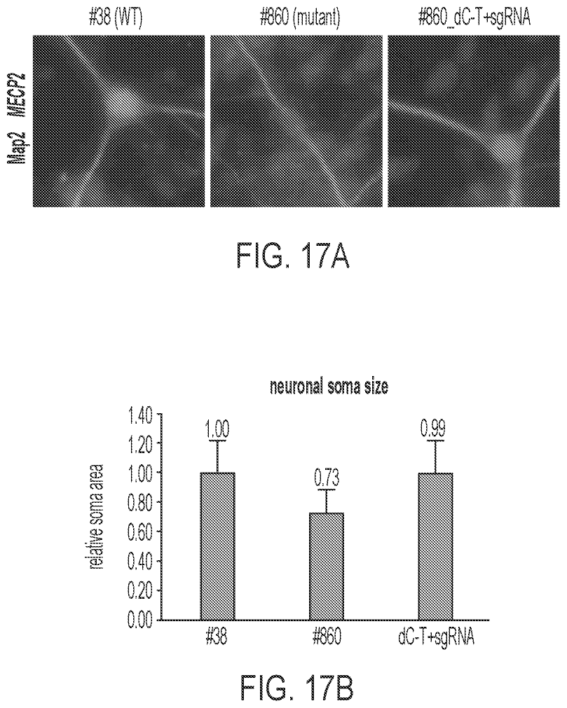

Neurons derived from wild type #38 hESC, mutant #860 RTT-like hESC, and methylation edited #860 were used to examine the soma size by immunofluorescence staining against MECP2 and Map2 (FIG. 17A). The soma sizes were quantified by Image J (FIG. 17B). The results show the rescue of neuronal soma size in methylation edited neurons. sgRNAs: 10 sgRNAs as discussed above. dC-T: dCas9-Tet1. dCas9 is Cas9 with the following point mutations: D10A and H840A.

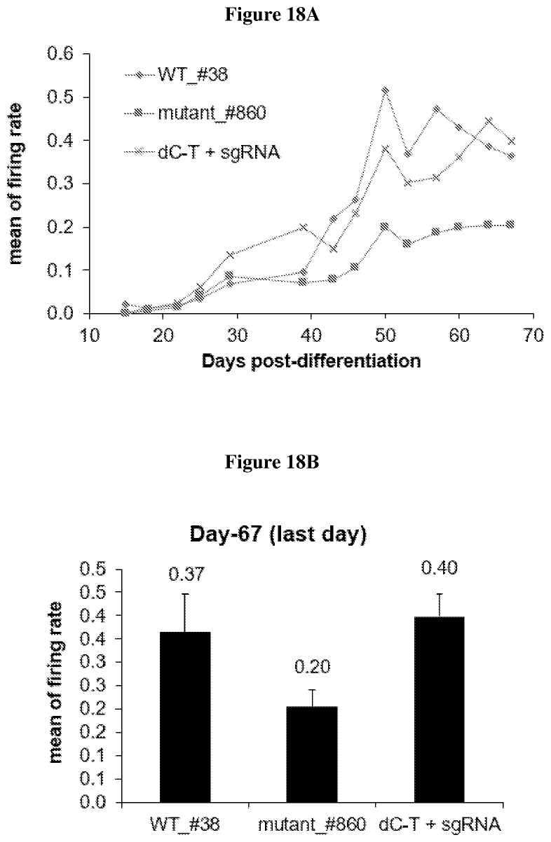

Neurons derived from wild type #38 hESC, mutant #860 RTT-like hESC, and methylation edited #860 were used to examine the electrophysical properties post-differentiation by multi-electrode assay (FIG. 18A). FIGS. 18A-18B show rescue of neuronal activity in methylation edited neurons. sgRNAs: 10 sgRNAs as discussed above. dC-T: dCas9-Tet1. dCas9 is Cas9 with the following point mutations: D10A and H840A.

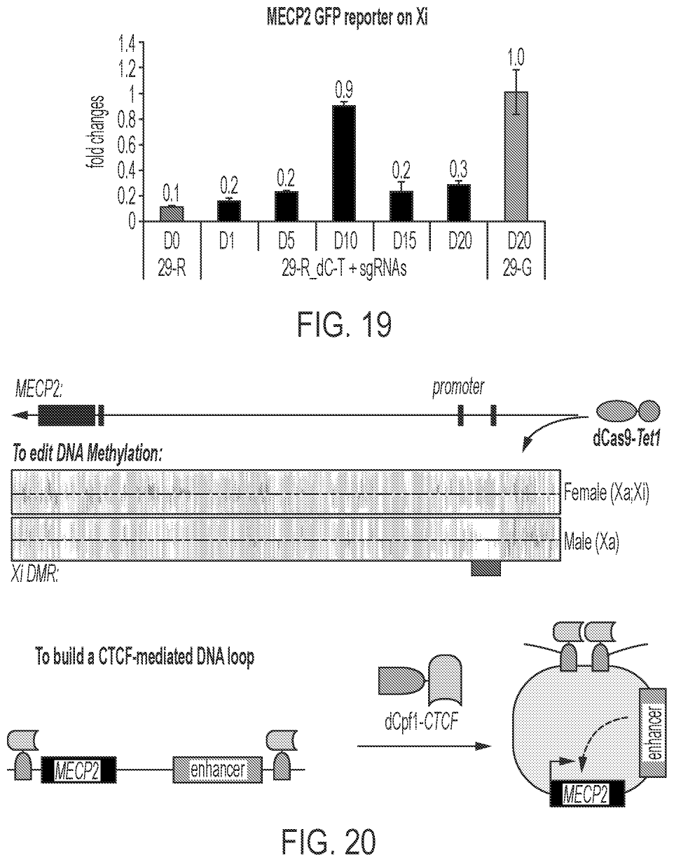

Neurons derived from wild type #38 hESC, mutant #860 RTT-like hESC, and methylation edited #860 were infected with lentiviral dCas9-Tet1 and 10 sgRNAs, and the expression of GFP was examined by qPCR. The results show that MECP2 reactivation was not stable in neurons (FIG. 19). sgRNAs: 10 sgRNAs as discussed above.

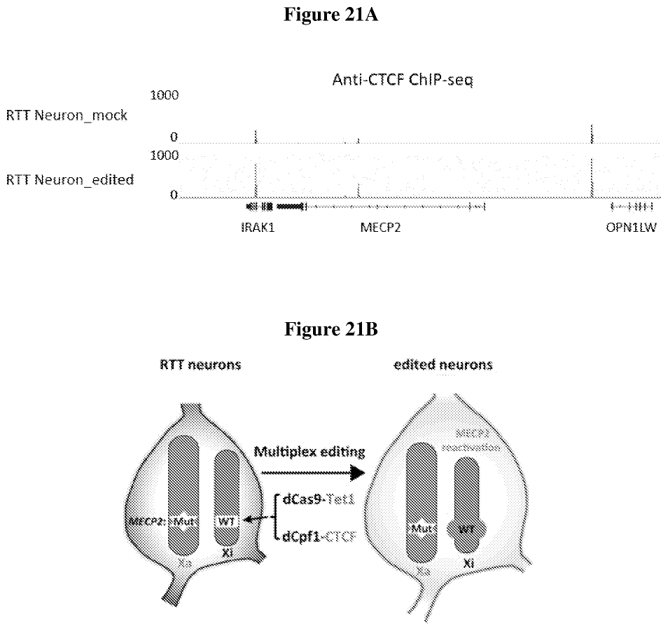

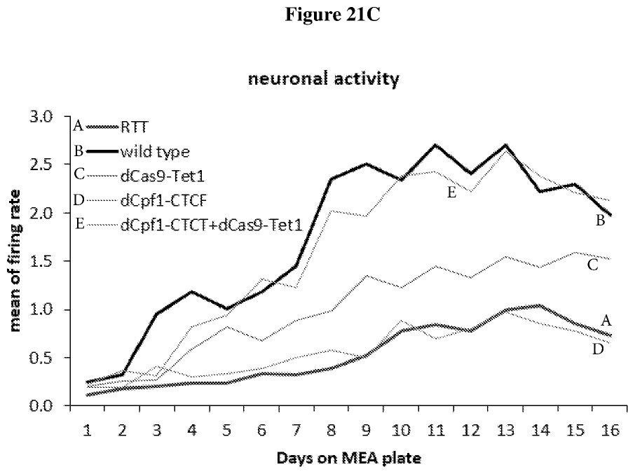

There are multiple layers of epigenetic mechanisms during X chromosome inactivation. dCpf1-CTCF was used to build an artificial escapee at the MECP2 locus on Xi for reactivation in neurons by dCas9-Tet1. FIGS. 21A-21C show that the combination of methylation editing and DNA looping in RTT neurons rescued the neuronal activity. dCas9 is Cas9 with the following point mutations: D10A and H840A; dCpf1 is LbCpf1 with the following point mutation: D833A.

### Methods

### Plasmid Design and Construction

PCR amplified Tea catalytic domain from pJFA344C7 (Addgene plasmid: 49236), Tea inactive catalytic domain from MLM3739 (Addgene plasmid: 49959), and tagBFP (synthesized gene block) were cloned into FUW vector (Addgene plasmid: 14882) with AscI, EcoRI and PfIMI to package lentiviruses. The target sgRNA expression plasmids were cloned by inserting annealed oligos into modified pgRNA plasmid (Addgene plasmid: 44248) with AarI site. A synthetic gBlock encoding the bacteriophage AcrIIA4 purchased from IDT was cloned into a modified FUW vector with AscI and EcoRI to package lentiviruses. All constructs were sequenced before transfection.

### Cell Culture and Lentivirus Production

iPSCs were cultured either with mTeSR1 medium (STEMCELL, #85850) or on irradiated mouse embryonic fibroblasts (MEFs) with standard hESCs medium: MMEM/F12 (Invitrogen) supplemented with 15% fetal bovine serum (GIBCO HI FBS, 10082-147), 5% KnockOut Serum Replacement (Invitrogen), 2 mM L-glutamine (MPBio), 1% nonessential amino acids (Invitrogen), 1% penicillin-streptomycin (Lonza), 0.1 mM b-mercaptoethanol (Sigma) and 4 ng/ml FGF2 (R&D systems)]. Lentiviruses expressing dCas9-Tet1-P2A-BFP, sgRNAs, and AcrIIA4 were produced by transfecting HEK293T cells with FUW constructs or pgRNA constructs together with standard packaging vectors (pCMV-dR8.74 and pCMV-VSVG) followed by ultra-centrifugation-based concentration. Virus titer (T) was calculated based on the infection efficiency for 293T cells, where T=(P*N)/(V), T=titer (TU/ul), p=% of infection positive cells according to the fluorescence marker, N=number of cells at the time of transduction, V=total volume of virus used. Note TU stands for transduction unit. Lentiviruses labeling NPCs (EF1A-GFP and EF1A-RFP) were purchased from Cellomics Technology.

### Multi-Electrode Array Recording

Two- or four-week-old differentiating neuronal cultures were dissociated using Accutase and 5×10⁵ cells were plated on each single well in the PEI-coated Axion Biosystems #M768-GL1-30Pt200 arrays. Recordings of spontaneous activities during a 5-minute period were performed on days indicated. Biological triplicates for each type of neurons were included.

### Immunocytochemistry, Immunohistochemistry, Microscopy, and Image Analysis

iPSCs and neurons were fixed with 4% paraformaldehyde (PFA) for 10 min at room temperature. Cells were permeabilized with PB ST (1×PBS solution with 0.1% Triton X-100) before blocking with 10% Normal Donkey Serum (NDS) in PBST. Cells were then incubated with appropriately diluted primary antibodies in PBST with 5% NDS for 1 hours at room temperature or 12 hours at 4° C., washed with PBST for 3 times at room temperature and then incubated with desired secondary antibodies in TBST with 5% NDS and DAPI to counter stain the nuclei. The following antibodies were used in this study: Chicken anti-GFP (1:1000, Ayes Labs), Rabbit anti-FMRP (1:50, Cell Signaling), Chicken anti-MAP2 (1:1000, Encor Biotech), Goat anti-mCherry (1:1000, SICGEN). Images were captured on a Zeiss LSM710 confocal microscope and processed with Zen software, ImageJ/Fiji, and Adobe Photoshop. For imaging-based quantification, unless otherwise specified, 3-5 representative images were quantified and data were plotted as mean±SD with Excel or Graphpad Prism.

### FACS Analysis

To isolate the infection-positive cell after lentiviral transduction, the treated cells were dissociated with trypsin and single-cell suspensions were prepared in growth medium subject to a BD FACSAria cell sorter according to the manufacture's protocol. Data were analyzed with FlowJo software.

### Western Blot

Cells were lysed by RIPA buffer with proteinase inhibitor (Invitrogen), and subject to standard immunoblotting analysis. Mouse anti-Cas9 (1:1000, Active Motif), mouse a-Tubulin (1:1000, Sigma), mouse anti-FMR1polyG (1:1000, EMD Millipore), rabbit anti-FMRP (1:100, Cell Signaling) antibodies were used.

### RT-qPCR

Cells were harvested using Trizol followed by Direct-zol (Zymo Research), according to manufacturer's instructions. RNA was converted to cDNA using First-strand cDNA synthesis (Invitrogen SuperScript III). Quantitative PCR reactions were prepared with SYBR Green (Invitrogen), and performed in 7900HT Fast ABI instrument.

### Chromatin Immunoprecipitation

Chromatin immunoprecipitation (ChIP) was performed as described in (Lee et al., 2006 Chromatin immunoprecipitation and microarray-based analysis of protein location. Nat. Protoc. 1, 729-748) with a few adaptations. Cells were crosslinked for 15 minutes at room temperature by the addition of one-tenth volume of fresh 11% formaldehyde solution (11% formaldehyde, 50 mM HEPES pH 7.3, 100 mM NaCl, 1 mM EDTA pH 8.0, 0.5 mM EGTA pH 8.0) to the growth media followed by 5 min quenching with 125 mM glycine. Cells were rinsed twice with 1×PBS and harvested using a silicon scraper and flash frozen in liquid nitrogen. Frozen crosslinked cells were stored at −80° C. For immunoprecipitation of lysate from 100 million cells, 50 ml of Protein G Dynabeads (Life Technologies #10009D) and 5 mg of antibody were prepared as follows. Dynabeads were washed 3× for 5 minutes with 0.5% BSA (w/v) in PBS. Magnetic beads were bound with the antibody overnight at 4° C., and then washed 3× with 0.5% BSA (w/v) in PBS.

Cells were prepared for ChIP as follows. All buffers contained freshly prepared 1×cOmplete protease inhibitors (Roche, 11873580001). Frozen crosslinked cells were thawed on ice and then resuspended in lysis buffer I (50 mM HEPES-KOH, pH 7.5, 140 mM NaCl, 1 mM EDTA, 10% glycerol, 0.5% NP-40, 0.25% Triton X-100, 1×protease inhibitors) and rotated for 10 minutes at 4° C., then spun at 1350 rcf. for 5 minutes at 4° C. The pellet was resuspended in lysis buffer II (10 mM Tris-HCl, pH 8.0, 200 mM NaCl, 1 mM EDTA, 0.5 mM EGTA, 1×protease inhibitors) and rotated for 10 minutes at 4° C. and spun at 1350 rcf. for 5 minutes at 4° C. The pellet was resuspend in sonication buffer (20 mM Tris-HCl pH 8.0, 150 mM NaCl, 2 mM EDTA pH 8.0, 0.1% SDS, and 1% Triton X-100, 1×protease inhibitors) and then sonicated on a Misonix 3000 sonicator for 10 cycles at 30 s each on ice (18-21 W) with 60 s on ice between cycles. Sonicated lysates were cleared once by centrifugation at 16,000 rcf. for 10 minutes at 4° C. 50 uL was reserved for input, and then the remainder was incubated overnight at 4° C. with magnetic beads bound with antibody to enrich for DNA fragments bound by the indicated factor.

Beads were washed twice with each of the following buffers: wash buffer A (50 mM HEPES-KOH pH 7.5, 140 mM NaCl, 1 mM EDTA pH 8.0, 0.1% Na-Deoxycholate, 1% Triton X-100, 0.1% SDS), wash buffer B (50 mM HEPES-KOH pH 7.9, 500 mM NaCl, 1 mM EDTA pH 8.0, 0.1% Na-Deoxycholate, 1% Triton X-100, 0.1% SDS), wash buffer C (20 mM Tris-HCl pH8.0, 250 mM LiCl, 1 mM EDTA pH 8.0, 0.5% Na-Deoxycholate, 0.5% IGEPAL C-630 0.1% SDS), wash buffer D (TE with 0.2% Triton X-100), and TE buffer. DNA was eluted off the beads by incubation at 65° C. for 1 hour with intermittent vortexing in 200 uL elution buffer (50 mM Tris-HCL pH 8.0, 10 mM EDTA, 1% SDS). Cross-links were reversed overnight at 65° C. To purify eluted DNA, 200 uL TE was added and then RNA was degraded by the addition of 2.5 mL of 33 mg/mL RNase A (Sigma, R4642) and incubation at 37° C. for 2 hours. Protein was degraded by the addition of 10 mL of 20 mg/mL proteinase K (Invitrogen, 25530049) and incubation at 55° C. for 2 hours. A phenol:chloroform:isoamyl alcohol extraction was performed followed by an ethanol precipitation. The DNA was then resuspended in 50 uL TE and used for sequencing. Purified ChIP DNA was used to prepare Illumina multiplexed sequencing libraries. Libraries for Illumina sequencing were prepared following the Illumina TruSeq DNA Sample Preparation v2 kit. Amplified libraries were size-selected using a 2% gel cassette in the Pippin Prep system from Sage Science set to capture fragments between 200 and 400 bp. Libraries were quantified by qPCR using the KAPA Biosystems Illumina Library Quantification kit according to kit protocols. Libraries were sequenced on the Illumina HiSeq 2500 for 40 bases in single read mode.

### Cas9 ChIP-Seq Peak Calling Method

Cas9 ChIP-seq data was analyzed as follows. Reads are de-multiplexed and mapped to human genome (hg19) using STAR (Dobin et al., STAR: ultrafast universal RNA-seq aligner. Bioinformatics, 2013, 29, 15-21), requiring unique mapping and perfect match. Peaks are called using MACS (Zhang et al., Model-based analysis of ChIP-seq (MACS), Genome Biol., 2008, 9, R137) with equal number of collapsed reads sampled to match sequencing depth.

### ChIP-BS-Seq

Anti-Cas9 ChIP experiment was performed as described above. The BS conversion and sequencing library preparation were performed according to the instructions by EpiNext High-Sensitivity Bisulfite-Seq Kit (EPIGENTEK, #P-1056A) and EpiNext NGS Barcode (EPIGENTEK, #P-1060). To analyze the raw data, the adaptor sequences in the illumina reads identified with FastQC were removed with Trim Galore. BS-Seq aligner Bismark (Krueger and Andrews, Bismark: a flexible aligner and methylation caller for Bisulfite-Seq applications, Bioinformatics, 2011, 27, 1571-1572) was used for assigning reads to human genome hg19 and calling methylation with bismark methylation extractor. To increase the number of uniquely mapped reads, after the first bismark alignment, 5 bases from the 50 and one base from the 30 of the unmapped reads were trimmed based on FastQC analysis. The resulting trimmed reads were then aligned to genome with Bismark. In both cases, bismark was ran with the options “-non directional-un-ambiguous-bowtie2-N 1-p 4-score min L,-6,-0.3—solexal.3-quals.” To compare the methylation levels of dCas9-Tet1 binding sites between dC-T and dC-dT samples, only the anti-Cas9 ChIP-seq peaks that included at least 20 CpG sites in which each CpG was covered with at least 10 reads in iPSCs and 5 reads in neurons by ChIP-BS-seq were selected to calculate the methylation levels. The number of binding sites in iPSC cells is 1018 and 670 in neurons. The scan for matches was utilized to search for the GGCGGCGGCGGCGGCGGCGGNGG motif in the sequences derived from those binding sites. R scripts were written for generating graphs.

### Bisulfite Conversion, PCR and Sequencing

Bisulfite conversion of DNA was established using the EpiTect Bisulfite Kit (QIAGEN) following the manufacturer's instructions. The resulting modified DNA was amplified by first round of nested PCR, following a second round using loci specific PCR primers. The first round of nested PCR was done as follows: 94° C. for 4 min; 55° C. for 2 min; 72° C. for 2 min; Repeat steps 1-3 1×; 94° C. for 1 min; 55° C. for 2 min; 72° C. for 2 min; Repeat steps 5-7 35×; 72° C. for 5 min; Hold 12° C. The second round of PCR was as follows: 95° C. for 4 min; 94° C. for 1 min; 55° C. for 2 min; 72° C. for 2 min; Repeat steps 2-4 35×; 72° C. for 5 min; Hold 12° C. The resulting amplified products were gel-purified, sub-cloned into a pCR2.1-TOPO-TA cloning vector (Life technologies), and sequenced.

### DNA Methylation Analysis

Pyro-seq of all bisulfite converted genomic DNA samples were performed with PyroMark Q48 Autoprep (QIAGEN) according to the manufacturer's instructions. Methylation analysis of CGG trinucleotide repeats: Methylation status of CGG repeats were analyzed by Claritas Genomics Inc. with Asuragen AmplideX_mPCR approach.

### Surveyor Assay

The ability of a gRNA, crRNA or sgRNA to direct sequence-specific binding of a CRISPR complex to a target sequence may be assessed by any suitable assay, such as by Surveyor assay.

Surveyor assay detects mutations and polymorphisms in a DNA mixture. Surveyor Nuclease can be a member of the CEL family of mismatch-specific nucleases derived from celery. Surveyor Nuclease recognizes and cleaves mismatches due to the presence of single nucleotide polymorphisms (SNPs) or small insertions or deletions. Surveyor nuclease cleaves with high specificity at the 3′ side of any mismatch site in both DNA strands, including all base substitutions and insertion/deletions up to at least 12 nucleotides.

The SURVEYOR nuclease cleaves with high specificity at the 3′ side of any mismatch site in both DNA strands, including all base substitutions and insertion/deletions up to at least 12 nucleotides. The Surveyor nuclease technology involves four steps: (i) PCR to amplify target DNA from the cell or tissue samples underwent Cas9/Cpf1 nuclease-mediated cleavage; (ii) hybridization to form heteroduplexes between affected and unaffected DNA (because the affected DNA sequence is different from the affected, a bulge structure resulted from the mismatch can form after denature and renature); (iii) treatment of annealed DNA with a Surveyor nuclease to cleave heteroduplexes (i.e., cut the bulges); and (iv) analysis of digested DNA products using the detection/separation platform of choice, for instance, agarose gel electrophoresis. The Cas9 nuclease-mediated cleavage efficacy can be estimated by the ratio of Surveyor nuclease-digested DNA to undigested DNA. The technology is highly sensitive, capable of detecting rare mutants present at as low as 1 in 32 copies. Surveyor mutation assay kits are commercially available from Integrated DNA Technologies (IDT), Coraville, IA.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A system comprising:
(a) a first polynucleotide sequence encoding a fusion protein comprising a deoxyribonuclease (DNase) dead Cpf1 (dCpf1) and an effector domain, wherein the dCpf1 is Cpf1 comprising (i) one or more of the following mutations: D908A, E993A, R1226A and D1263A, or (ii) the following mutation: D833A; and
(b) a second polynucleotide sequence encoding one or more guide sequences that hybridize to one or more target sequences.

2. The system of claim 1, wherein the one or more guide sequences is/are one or more CRISPR RNA (crRNA) molecules, one or more single-guide RNA (sgRNA) molecules, one or more guide RNA (gRNA) molecules, or combinations thereof.

3. The system of claim 1, wherein the first polynucleotide sequence and the second polynucleotide sequence are on a single vector.

4. The system of claim 1, wherein the first polynucleotide sequence and the second polynucleotide sequence are on different vectors.

5. The system of claim 1, wherein the second polynucleotide sequence encodes two or more crRNA molecules that hybridize to two or more target sequences.

6. The system of claim 1, wherein the dCpf1 has ribonuclease (RNase) activity.

7. The system of claim 1, wherein the effector domain is TET2, Dnmt3b or CTCF.

8. The system of claim 1, wherein the effector domain has an activity to modify an epigenome.

9. The system of claim 1, wherein the effector domain is an enzyme that modifies a histone subunit.

10. The system of claim 1, wherein the effector domain is a histone acetyltransferase (HAT), histone deacetylase (HDAC), histone methyltransferase (HMT), or histone demethylase.

11. The system of claim 10, wherein the HAT is p300.

12. The system of claim 1, wherein the effector domain is an enzyme that modifies methylation state of DNA.

13. The system of claim 1, wherein the effector domain is a DNA methyltransferase (DNMT) or a Ten-Eleven-Translocation (TET) methylcytosine dioxygenase protein.

14. The system of claim 13, wherein the DNMT protein is Dnmt3b.

15. The system of claim 13, wherein the TET protein is Tet2.

16. The system of claim 1, wherein the effector domain is CTCF.

17. The system of claim 16, wherein the CTCF is wild type CTCF or a DNA binding mutant CTCF.

18. The system of claim 17, wherein the DNA binding mutant CTCF comprises one or more of the following mutations: K365A, R368A, R396A, and Q418A.

19. The system of claim 1, wherein the effector domain is a transcriptional activation domain.

20. The system of claim 19, wherein the transcriptional activation domain is derived from VP64 or NF-κB p65.

21. The system of claim 1, wherein the effector domain is a transcriptional silencer or transcriptional repression domain.

22. The system of claim 21, wherein the transcriptional repression domain is a Krueppel-associated box (KRAB) domain, ERF repressor domain (ERD), or mSin3A interaction domain (SID).

23. The system of claim 21, wherein the transcriptional silencer is heterochromatin protein 1 (HP1), or Methyl CpG binding Protein 2 (MeCP2).

24. The system of claim 1, wherein the Cpf1 is from Flavobacterium brachiophilum, Parcubacteria bacterium, Peregrinibacteria bacterium, Acidaminococcus sp., Porphyromonas macacae, Lachnospiraceae bacterium, Porphyromonas crevioricanis, Prevotella disiens, Moraxella bovoculi, Leptospira inadai, Lachnospiraceae bacterium (MA2020), Francisella novicida, Candidatus methanoplasma termitum, or Eubacterium eligens.

25. A composition comprising the system of claim 1.

26. A cell comprising the system of claim 1.

27. One or more vectors comprising the system of claim 1.

28. The one or more vectors of claim 27, wherein the one or more vectors comprise a recombinant lentiviral vector.

29. A method for modifying an epigenome of a cell, the method comprising contacting the cell with the system of claim 1.

30. A method for modifying an epigenome of a cell, the method comprising contacting the cell with a system comprising:
(a) a first polynucleotide sequence encoding a fusion protein comprising a deoxyribonuclease (DNase) dead Cpf1 (dCpf1) and an effector domain, wherein the dCpf1 is Cpf1 comprising (i) one or more of the following mutations: D908A, E993A, R1226A and D1263A, or (ii) the following mutation: D833A; and
(b) a second polynucleotide sequence encoding one or more guide sequences that hybridize to one or more target sequences.

31. The method of claim 30, wherein the first polynucleotide sequence and the second polynucleotide sequence are on a single vector.

32. A method for treating a disease in a patient, the method comprising administering to the patient a system comprising:
(a) a first polynucleotide sequence encoding a fusion protein comprising a deoxyribonuclease (DNase) dead Cpf1 (dCpf1) and an effector domain, wherein the dCpf1 is Cpf1 comprising (i) one or more of the following mutations: D908A, E993A, R1226A and D1263A, or (ii) the following mutation: D833A; and
(b) a second polynucleotide sequence encoding one or more guide sequences that hybridize to one or more target sequences.

33. The method of claim 32, wherein the first polynucleotide sequence and the second polynucleotide sequence are on a single vector.

34. The method of claim 32, wherein the one or more target sequences are in one or more genes selected from the group consisting of: MECP2, PHEX, COL4A5, COL4A3, COL4A1, IKBKG, PORCN, DMD/DYS, RPS6KA3, LAMP2, NSDHL, PDHA1, HDAC8, SMC1A, CDKL5, OFD1, WDR45, KDM6A, CASK, FINA, ALAS2, HNRNPH2, MSL3 and IQSEC2.

35. The method of claim 32, wherein the one or more target sequences are in one or more genes selected from Table 1 or Table 2.

36. The method of claim 32, wherein the disease is a X-linked disease.

37. The method of claim 36, wherein the X-linked disease is selected from Table 1.

38. The method of claim 32, wherein the disease is an imprinting-related disease.

39. The method of claim 30, wherein the cell is an induced pluripotent stem cell (iPSC) or a human embryonic stem cell (hESC).

40. The method of claim 39, wherein the iPSC is derived from a fibroblast of a subject.

41. The method of claim 39, further comprising culturing the iPSC to differentiate into a neuron.

42. The method of claim 41, further comprising administering the neuron to a subject.


SEQUENCE LISTING (44 sequences)

SEQ ID NO: 1 - RNA, 20 nt, synthetic construct
  misc_feature 1..20: note = Description of Artificial Sequence: Synthetic oligonucleotide
  source 1..20: mol_type = other RNA
taatttctactcttgtagat

SEQ ID NO: 2 - DNA, 42 bp, synthetic construct
  misc_feature 1..42: note = Description of Artificial Sequence: Synthetic oligonucleotide
  source 1..42: mol_type = other DNA
ttaatgtttcctgatggtccatgtctgttactcgcctgtcaa

SEQ ID NO: 3 - DNA, 42 bp, synthetic construct
  misc_feature 1..42: note = Description of Artificial Sequence: Synthetic oligonucleotide
  source 1..42: mol_type = other DNA
tccctctttgctaggaatattgaagggggcaggggaaggcgg

SEQ ID NO: 4 - DNA, 42 bp, synthetic construct
  misc_feature 1..42: note = Description of Artificial Sequence: Synthetic oligonucleotide
  source 1..42: mol_type = other DNA
gttgggtttggtgctcaatgaaaggagataaggtccttgaat

SEQ ID NO: 5 - RNA, 22 nt, synthetic construct
  misc_feature 1..22: note = Description of Artificial Sequence: Synthetic oligonucleotide
  source 1..22: mol_type = other RNA
aatttctactcttgtagatgag

SEQ ID NO: 6 - RNA, 40 nt, synthetic construct
  misc_feature 1..40: note = Description of Artificial Sequence: Synthetic oligonucleotide
  source 1..40: mol_type = other RNA
gtctaagaactttaaataattttctactgttgtagaagag

SEQ ID NO: 7 - protein, 27 aa, synthetic construct
  REGION 1..27: note = Description of Artificial Sequence: Synthetic peptide
GGGGSPKKKRKVGPKKKRKVDGGGGSE

SEQ ID NO: 8 - DNA, 81 bp, synthetic construct
  misc_feature 1..81: note = Description of Artificial Sequence: Synthetic oligonucleotide
  source 1..81: mol_type = other DNA
ggtggcggaggctcgccaaaaaagaagagaaaggtaggtccaaagaaaaaacgaaaagtagatggtggcggaggatccgaa

SEQ ID NO: 9 - DNA, 54 bp, synthetic construct
  misc_feature 1..54: note = Description of Artificial Sequence: Synthetic oligonucleotide
  source 1..54: mol_type = other DNA
taaaaaaattggctctccggcacgccctttcatctacaagagtagaaattgacg

SEQ ID NO: 10 - DNA, 60 bp, synthetic construct
  misc_feature 1..60: note = Description of Artificial Sequence: Synthetic oligonucleotide
  source 1..60: mol_type = other DNA
ctagcgtcaatttctactcttgtagatgaaagggcgtgccggagagccaatttttttaat

SEQ ID NO: 11 - DNA, 773 bp, synthetic construct
  misc_feature 1..773: note = Description of Artificial Sequence: Synthetic polynucleotide
  source 1..773: mol_type = other DNA
atttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctgg

SEQ ID NO: 12 - DNA, 25 bp, synthetic construct
  misc_feature 1..25: note = Description of Artificial Sequence: Synthetic oligonucleotide
  source 1..25: mol_type = other DNA
tcctccttccttgccaacgctggct

SEQ ID NO: 13 - DNA, 22 bp, synthetic construct
  misc_feature 1..22: note = Description of Artificial Sequence: Synthetic oligonucleotide
  source 1..22: mol_type = other DNA
gctggcaggcgggggagcgcgg

SEQ ID NO: 14 - DNA, 24 bp, synthetic construct
  misc_feature 1..24: note = Description of Artificial Sequence: Synthetic oligonucleotide
  source 1..24: mol_type = other DNA
ttggagcagcaaagttgcccaccc

SEQ ID NO: 15 - DNA, 24 bp, synthetic construct
  misc_feature 1..24: note = Description of Artificial Sequence: Synthetic oligonucleotide
  source 1..24: mol_type = other DNA
aaacgggtgggcaactttgctgct

SEQ ID NO: 16 - DNA, 24 bp, synthetic construct
  misc_feature 1..24: note = Description of Artificial Sequence: Synthetic oligonucleotide
  source 1..24: mol_type = other DNA
ttggtagtgatattgagaaaatgt

SEQ ID NO: 17 - DNA, 24 bp, synthetic construct
  misc_feature 1..24: note = Description of Artificial Sequence: Synthetic oligonucleotide
  source 1..24: mol_type = other DNA
aaacacattttctcaatatcacta

SEQ ID NO: 18 - DNA, 24 bp, synthetic construct
  misc_feature 1..24: note = Description of Artificial Sequence: Synthetic oligonucleotide
  source 1..24: mol_type = other DNA
ttggcagccaatcaacagctggag

SEQ ID NO: 19 - DNA, 24 bp, synthetic construct
  misc_feature 1..24: note = Description of Artificial Sequence: Synthetic oligonucleotide
  source 1..24: mol_type = other DNA
aaacctccagctgttgattggctg

SEQ ID NO: 20 - DNA, 22 bp, synthetic construct
  misc_feature 1..22: note = Description of Artificial Sequence: Synthetic oligonucleotide
  source 1..22: mol_type = other DNA
ttgggccatcacagccaatgac

SEQ ID NO: 21 - DNA, 22 bp, synthetic construct
  misc_feature 1..22: note = Description of Artificial Sequence: Synthetic oligonucleotide
  source 1..22: mol_type = other DNA
aaacgtcattggctgtgatggc

SEQ ID NO: 22 - DNA, 23 bp, synthetic construct
  misc_feature 1..23: note = Description of Artificial Sequence: Synthetic oligonucleotide
  source 1..23: mol_type = other DNA
ttggaggaggagagactgtgagt

SEQ ID NO: 23 - DNA, 23 bp, synthetic construct
  misc_feature 1..23: note = Description of Artificial Sequence: Synthetic oligonucleotide
  source 1..23: mol_type = other DNA
aaacactcacagtctctcctcct

SEQ ID NO: 24 - DNA, 24 bp, synthetic construct
  misc_feature 1..24: note = Description of Artificial Sequence: Synthetic oligonucleotide
  source 1..24: mol_type = other DNA
ttggggagggggagggtagagagg

SEQ ID NO: 25 - DNA, 24 bp, synthetic construct
  misc_feature 1..24: note = Description of Artificial Sequence: Synthetic oligonucleotide
  source 1..24: mol_type = other DNA
aaaccctctctaccctccccctcc

SEQ ID NO: 26 - DNA, 22 bp, synthetic construct
  misc_feature 1..22: note = Description of Artificial Sequence: Synthetic oligonucleotide
  source 1..22: mol_type = other DNA
ttgggggaggaagaggggcgtc

SEQ ID NO: 27 - DNA, 22 bp, synthetic construct
  misc_feature 1..22: note = Description of Artificial Sequence: Synthetic oligonucleotide
  source 1..22: mol_type = other DNA
aaacgacgcccctcttcctccc

SEQ ID NO: 28 - DNA, 24 bp, synthetic construct
  misc_feature 1..24: note = Description of Artificial Sequence: Synthetic oligonucleotide
  source 1..24: mol_type = other DNA
ttggtgagagctcaggagcccttg

SEQ ID NO: 29 - DNA, 24 bp, synthetic construct
  misc_feature 1..24: note = Description of Artificial Sequence: Synthetic oligonucleotide
  source 1..24: mol_type = other DNA
aaaccaagggctcctgagctctca

SEQ ID NO: 30 - DNA, 24 bp, synthetic construct
  misc_feature 1..24: note = Description of Artificial Sequence: Synthetic oligonucleotide
  source 1..24: mol_type = other DNA
ttggcctacttgttcctgctagat

SEQ ID NO: 31 - DNA, 24 bp, synthetic construct
  misc_feature 1..24: note = Description of Artificial Sequence: Synthetic oligonucleotide
  source 1..24: mol_type = other DNA
aaacatctagcaggaacaagtagg

SEQ ID NO: 32 - DNA, 24 bp, synthetic construct
  misc_feature 1..24: note = Description of Artificial Sequence: Synthetic oligonucleotide
  source 1..24: mol_type = other DNA
ttggaggtggttatagttcccatc

SEQ ID NO: 33 - DNA, 24 bp, synthetic construct
  misc_feature 1..24: note = Description of Artificial Sequence: Synthetic oligonucleotide
  source 1..24: mol_type = other DNA
aaacgatgggaactataaccacct

SEQ ID NO: 34 - DNA, 18 bp, synthetic construct
  misc_feature 1..18: note = Description of Artificial Sequence: Synthetic oligonucleotide
  source 1..18: mol_type = other DNA
gagggggagggtagagag

SEQ ID NO: 35 - DNA, 30 bp, synthetic construct
  misc_feature 1..30: note = Description of Artificial Sequence: Synthetic oligonucleotide
  source 1..30: mol_type = other DNA
ctccctcctctccaaaaaaaaactataata

SEQ ID NO: 36 - DNA, 15 bp, synthetic construct
  misc_feature 1..15: note = Description of Artificial Sequence: Synthetic oligonucleotide
  source 1..15: mol_type = other DNA
gggagggtagagagg

SEQ ID NO: 37 - DNA, 21 bp, synthetic construct
  misc_feature 1..21: note = Description of Artificial Sequence: Synthetic oligonucleotide
  source 1..21: mol_type = other DNA
gggtagaggggggtagaaatt

SEQ ID NO: 38 - DNA, 20 bp, synthetic construct
  misc_feature 1..20: note = Description of Artificial Sequence: Synthetic oligonucleotide
  source 1..20: mol_type = other DNA
acccccacctctccctaaat

SEQ ID NO: 39 - DNA, 19 bp, synthetic construct
  misc_feature 1..19: note = Description of Artificial Sequence: Synthetic oligonucleotide
  source 1..19: mol_type = other DNA
agagtttaggagtttttgt

SEQ ID NO: 40 - DNA, 26 bp, synthetic construct
  misc_feature 1..26: note = Description of Artificial Sequence: Synthetic oligonucleotide
  source 1..26: mol_type = other DNA
gagttgtgggatttagaatataatgt

SEQ ID NO: 41 - DNA, 27 bp, synthetic construct
  misc_feature 1..27: note = Description of Artificial Sequence: Synthetic oligonucleotide
  source 1..27: mol_type = other DNA
ctccttctcccccattccataaatttc

SEQ ID NO: 42 - DNA, 16 bp, synthetic construct
  misc_feature 1..16: note = Description of Artificial Sequence: Synthetic oligonucleotide
  source 1..16: mol_type = other DNA
gttagatggggaaagg

SEQ ID NO: 43 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 44 - protein, 1228 aa, unidentified
  source 1..1228: note = Lachnospiraceae bacterium
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK